(19) United States
(12) Patent Application Publication
(10) Pub. No.: US 2021/0285944 A1
(43) Pub. Date: Sep. 16, 2021

(54) METHOD TO ENCAPSULATE AND PRESERVE IMMOBILIZED PROTEIN

(71) Applicants: Srikanth Singamaneni, St. Louis, MO (US); Rohit Gupta, St. Louis, MO (US); Jingyi Luan, St. Louis, MO (US); Shantanu Chakrabartty, St. Louis, MO (US); Erica Scheller, St. Louis, MO (US); Jeremiah Morrissey, St. Louis, MO (US); Yixuan Wang, St. Louis, MO (US); Zheyu Wang, St. Louis, MO (US)

(72) Inventors: Srikanth Singamaneni, St. Louis, MO (US); Rohit Gupta, St. Louis, MO (US); Jingyi Luan, St. Louis, MO (US); Shantanu Chakrabartty, St. Louis, MO (US); Erica Scheller, St. Louis, MO (US); Jeremiah Morrissey, St. Louis, MO (US); Yixuan Wang, St. Louis, MO (US); Zheyu Wang, St. Louis, MO (US)

(73) Assignee: Washington University, St. Louis, MO (US)

(21) Appl. No.: 17/198,823

(22) Filed: Mar. 11, 2021

Related U.S. Application Data
(60) Provisional application No. 62/988,649, filed on Mar. 12, 2020.

(51) Int. Cl. G01N 33/543

(52) U.S. Cl. CPC G01N 33/54346; G01N 33/5436

(57) ABSTRACT

The present disclosure is directed to refreshable biosensors and methods for synthesizing and refreshing same. In some embodiments, the refreshable biosensor comprises a plasmonic nanoparticle and a biorecognition element, wherein the biorecognition element is encapsulated with at least one of an organosilica polymer layer or a metal organic framework (MOF).

### CROSS-REFERENCE TO RELATED APPLICATIONS

This application claims priority to U.S. Provisional Application No. 62/988,649, filed Mar. 12, 2020, the content of which is incorporated herein by reference in its entirety.

### STATEMENT REGARDING FEDERALLY SPONSORED RESEARCH & DEVELOPMENT

This invention was made with government support under DE027098 and CA141521 awarded by the National Institutes of Health. The government has certain rights in the invention.

### BACKGROUND OF THE DISCLOSURE

Wearable and implantable biosensors have attracted extensive attention owing to their ability to provide continuous monitoring of biophysical and biochemical parameters in biofluids such as sweat, saliva, interstitial fluid, and tears. In the past few years, the frontiers and the possible applications of such devices are rapidly advancing from tracking physical activity and biophysical parameters to continuous monitoring of target molecular biomarkers at physiological and pathological concentrations. While there have been significant efforts in realizing wearable and implantable biosensors, there are still significant challenges that need to be overcome before such classes of biosensors are widely used for making timely clinical interventions in pathological conditions that rapidly manifest into life-threating events or chronic conditions. In fact, to date, only minimally invasive glucose monitoring devices have some commercial presence.

Biosensors designed for continuous monitoring of biochemical analytes should be able to detect and quantify the target analyte (e.g., biomarker) for an extended duration. However, the number of analyte binding sites in most biosensors are limited, and once saturated with target analytes, it becomes insensitive to further changes in the concentration of the analyte as the analyte-recognition element interactions (especially antibody-antigen interactions) are virtually irreversible under normal conditions. Additionally, the long-term usage of the biosensor is limited by the poor stability of antibodies. A possible approach to overcome these problems is to design a strategy to preserve the biorecognition elements and refresh the sensor without compromising the sensitivity and specificity of the biorecognition elements. As described herein, plasmonic nanostructures are employed as a transduction platform. Owing to their high refractive index sensitivity, plasmonic nanostructures are able to transduce biomolecular binding events (capture/release of the analyte) into a measurable shift within the localized surface plasmon resonance (LSPR) wavelength. The refractive index sensitivity of plasmonic nanostructures has been harnessed to realize various chemical and biological sensors. Plasmonic biosensors relying on antibodies as recognition elements are highly promising as lab-on-chip devices for label-free protein detection in point-of-care and resource-limited settings.

In most immunosensors, antigens are recognized and captured by antibodies via noncovalent interactions such as hydrogen bonds, van der Waals forces, electrostatic and hydrophobic interactions. These interactions are disrupted by extremes of pH, high salt concentrations and surfactants. Under these harsh conditions, natural antibodies are unstable and are prone to lose their biorecognition capability owing to their poor chemical and environmental stability. Various methods have been reported to preserve the biorecognition capability of antibodies under harsh conditions, which include encapsulation of immobilized antibodies with metallic-organic frameworks, silk, sucrose, or addition of other preservatives. While these strategies successfully preserve the biorecognition capability of biodiagnostic reagents immobilized on plasmonic nanotransducers against harsh environmental conditions during storage and transportation, they do not protect the antibodies during sensor operation or during sensor refreshing.

Further, COVID-19, an infectious disease caused by severe acute respiratory syndrome coronavirus-2 (SARS-CoV-2), has become a global public health challenge. As of January 2021, the disease has rapidly spread to more than 100 countries with over 85 million confirmed cases and nearly 1.8 million deaths. Accurate, fast and low-cost serologic assays, evaluating the presence of specific antibodies against the virus in the blood, facilitate the diagnosis and screening of symptomatic and asymptomatic patients, monitoring of the disease course and identification of possible convalescent serum donors in resource-limited regions.

Enzyme-linked immunosorbent assays (ELISA) is the most common method employed in serologic testing. ELISA involves surface immobilized antigens on microtiter plates to capture the SARS-CoV-2 antibodies in patient samples. The accuracy and reliability of ELISA critically depends on the structural integrity and biofunctionality of these biomolecules. However, due to the poor stability of proteins under ambient and elevated temperatures, both antibodies and antigens are prone to lose their structure and functionalities. More importantly, antigens immobilized on solid surface (e.g. microtiter plate) exhibit lower stability under non-refrigerated conditions compared to those in buffer solution. Therefore, “cold-chain” system is necessary to maintain the stability and ensure the performance of these assays following the storage, transportation, and handling of the diagnostic reagents. Unfortunately, besides the extra financial burden, cold chain systems are not feasible in developing parts of the world and resource-limited settings, where refrigeration and electricity are not available, but disease surveillance and control are critically needed. Therefore, it is imperative to develop a low-cost and facile, refrigeration-free technology to preserve the biorecognition capability of antigens immobilized on solid surface and disease-specific antibodies in patient samples, providing reliable and accurate serologic assays for resource-limited settings.

Metal-organic frameworks (MOFs), comprised of polynuclear metal clusters or ions bridged by organic ligands, are of increasing interest. MOFs exhibit extremely large surface area, tunable porosity, diverse chemical functionality and high thermal stability, making them highly attractive for biomineralization, encapsulation, biosensors, drug delivery, gas storage, and catalysis. Among these applications, of particular interest is the encapsulation of biomolecules via in situ growth of MOF crystals in the presence of biomolecules at room temperature under mild aqueous conditions. MOFs serve as rigid exoskeletons, preserving the structure and biofunctionality of embedded molecules against denaturation/degradation under elevated temperature, organic solvents, and proteolytic conditions. Although MOF encapsulation has been demonstrated for preserving enzymes, soluble proteins/biomarkers and antibodies in biosensors, preserving immobilized antigens in an immunoassay has not been demonstrated. In contrast to antibodies, antigens are more sensitive to environmental conditions after surface immobilization, necessitating effective biopreservation methods. Changes in their secondary and tertiary structure result in the loss of conformational epitopes and consequently the antibody recognition, thus compromising the accuracy and sensitivity of the assay. MOFs are a promising class of materials for preserving the structure and biofunctionality of surface-bound antigens (i.e. recognition elements) and antibodies (i.e. target analytes) in biospecimen, thus enabling reliable and accurate SARS-CoV-2 serologic assays even in resource-limited regions.

Accordingly, there is a need for stable, refreshable biosensors for long-term biomarker monitoring applications, including SARS-CoV-2 antibody and SARS-CoV-2 antigen applications. The embodiments described herein resolve at least these known deficiencies.

### BRIEF DESCRIPTION OF THE DISCLOSURE

In one aspect, the present disclosure is directed to a biosensor comprising a plasmonic nanostructure and at least one biorecognition element, wherein the biorecognition element is encapsulated with at least one of an organosilica polymer layer or a metal organic framework (MOF).

In some embodiments, the MOF comprises zeolitic imidazolate framework-90 (ZIF-90). In some embodiments, the at least one organosilica polymer layer comprises (3-aminopropyl) trimethoxysilane (APTMS) monomers, trimethoxy(propyl)silane (TMPS) monomers, or a combination thereof In some embodiments, the biorecognition element is anchored to the plasmonic nanostructure via a flexible linker comprising poly(ethylene glycol) (PEG). In some embodiments, the plasmonic nanostructure is selected from the group consisting of a gold nanorod and a gold nanorattle. In some embodiments, the biorecognition element is an antibody. In some embodiments, the biorecognition element is an antigen.

In another aspect, the present disclosure is directed to a method for synthesizing a refreshable biosensor. The method comprises immobilizing at least one biorecognition element on a plasmonic nanostructure, and encapsulating the at least one biorecognition element with at least one of an organosilica polymer layer via in situ polymerization or a metal organic framework via in situ crystallization.

In some embodiments, the MOF comprises zeolitic imidazolate framework-90 (ZIF-90). In some embodiments, the at least one organosilica polymer layer comprises (3-aminopropyl) trimethoxysilane (APTMS) monomers, trimethoxy(propyl)silane (TMPS) monomers, or a combination thereof In some embodiments, the biorecognition element is anchored to the plasmonic nanostructure via a flexible linker comprising poly(ethylene glycol) (PEG). In some embodiments, the plasmonic nanostructure is selected from the group consisting of a gold nanorod and a gold nanorattle. In some embodiments, the biorecognition element is an antibody. In some embodiments, the biorecognition element is an antigen.

In yet another aspect, the present disclosure is directed to a method for refreshing a biosensor. The method comprises exposing a biosensor to a target analyte, wherein the biosensor comprises at least one biorecognition element immobilized on a plasmonic nanostructure, and wherein the at least one biorecognition element is encapsulated with at least one of an organosilica polymer layer or a metal organic framework (MOF). The method further comprises rinsing the biosensor with an aqueous sodium dodecyl sulfate solution and re-exposing the biosensor to the target analyte.

In some embodiments, the MOF comprises zeolitic imidazolate framework-90 (ZIF-90). In some embodiments, the at least one organosilica polymer layer comprises (3-aminopropyl) trimethoxysilane (APTMS) monomers, trimethoxy(propyl)silane (TMPS) monomers, or a combination thereof In some embodiments, the biorecognition element is anchored to the plasmonic nanostructure via a flexible linker comprising poly(ethylene glycol) (PEG). In some embodiments, the plasmonic nanostructure is selected from the group consisting of a gold nanorod and a gold nanorattle. In some embodiments, the biorecognition element is an antibody. In some embodiments, the biorecognition element is an antigen.

### BRIEF DESCRIPTION OF THE DRAWINGS

The embodiments described herein may be better understood by referring to the following description in conjunction with the accompanying drawings.

FIG. 1A is an exemplary embodiment of an organosilica encapsulation of biorecognition elements for a refreshable biosensor in accordance with the present disclosure.

FIG. 1B is an exemplary embodiment of representative TEM image of AuNRs used as plasmonic nanotransducers in accordance with the present disclosure.

FIG. 1C is an exemplary embodiment of normalized vis-NIR extinction spectra of AuNR and AuNR conjugated with IgG, depicting an ˜8 nm red shift in the LSPR wavelength in accordance with the present disclosure.

FIG. 1D is an exemplary embodiment of a representative atomic force microscopy (AFM) image showing uniform distribution of AuNR-IgG bioconjugates on a glass substrate in accordance with the present disclosure.

FIG. 1E is an exemplary embodiment of AFM images of AuNR-IgG bioconjugates uniformly adsorbed on a glass substrate with no signs of aggregation or patchiness in accordance with the present disclosure.

FIG. IF is an exemplary embodiment of an LSPR shift of AuNR-IgG bioconjugates on glass substrates upon exposure to various concentrations of anti-IgG showing monotonic increase in the LSPR shift with concentration in accordance with the present disclosure. Error bars represent standard deviations from three different samples.

FIG. 1G is an exemplary embodiment of a schematic illustration of steps involved in the fabrication of the refreshable sensor in accordance with the present disclosure.

FIG. 1H is an exemplary embodiment of extinction spectra of AuNR-IgG bioconjugates obtained after each fabrication step shown in FIG. 1G. The inset shows zoomed-in spectra highlighting the shifts in the LSPR wavelength in accordance with the present disclosure.

FIG. 1I is an exemplary embodiment of LSPR shifts corresponding to biodetection and sensor refreshing in accordance with the present disclosure.

FIG. 1J is an exemplary embodiment of LSPR shift upon exposure of plasmonic biochips to different concentrations of anti-IgG before (black) and after sodium dodecyl sulfate (SDS) (red) treatment. Error bars represent standard deviations from three different samples in accordance with the present disclosure.

FIG. 1K is an exemplary embodiment of (%) retained biorecognition capability of AuNR-IgG bioconjugates measured over multiple anti-IgG capture/release cycles in accordance with the present disclosure. Error bars represent standard deviations from three different samples.

FIG. 2A is an exemplary embodiment of a schematic illustration of the steps involved in the organosilica-based biopreservation of bioconjugates to realize refreshable biosensors in accordance with the present disclosure.

FIG. 2B is an exemplary embodiment of an LSPR wavelength shift after exposure of AuNR-IgG bioconjugates to different concentrations of APTMS and TMPS monomers to achieve polymer encapsulation in accordance with the present disclosure. Error bars represent standard deviations from three different samples.

FIG. 2C is an exemplary embodiment of an AFM image of polymer encapsulated AuNR-IgG bioconjugates on glass substrates in accordance with the present disclosure.

FIG. 2D is an exemplary embodiment of a representative AFM image of AuNR-IgG bioconjugates on the glass substrate before (top) and after (bottom) polymer encapsulation with optimum monomer concentration (0.8 mg/mL) in accordance with the present disclosure.

FIG. 2E is an exemplary embodiment of a surface-enhanced Raman scattering spectra of AuNR-IgG bioconjugates before and after polymerization in accordance with the present disclosure.

FIG. 2F is an exemplary embodiment of the biorecognition capability corresponding to different monomer concentrations, which determines the polymer thickness in accordance with the present disclosure. Error bars represent standard deviations from three different samples.

FIG. 2G is an exemplary embodiment of a maximum LSPR shift obtained upon exposure of the AuNR-IgG biosensors, encapsulated with different polymerization conditions, to anti-IgG in accordance with the present disclosure.

FIG. 2H is an exemplary embodiment of the retained biorecognition capability of AuNR-IgG bioconjugates corresponding to different monomer concentrations, after SDS treatment in accordance with the present disclosure. Error bars represent standard deviations from three different samples.

FIG. 2I is an exemplary embodiment of an LSPR shift of the polymer-encapsulated biosensor after treatment with human serum albumin (HSA) and anti-IgG in accordance with the present disclosure. Error bars represent standard deviations from three different samples.

FIG. 2J is an exemplary embodiment of a normalized extinction spectra of PEGylated AuNR-IgG bioconjugates upon exposure to human serum albumin (HSA) (left) and anti-IgG (right) depicting significantly low non-specific binding of HSA even at high concentration in accordance with the present disclosure.

FIG. 2K is an exemplary embodiment of an LSPR shift of polymer encapsulated AuNR-IgG bioconjugates upon exposure to various concentrations of anti-IgG showing monotonic increase in the LSPR shift with concentration in accordance with the present disclosure. Error bars represent standard deviations from three different samples.

FIG. 3A is an exemplary embodiment of an AuNR extinction spectra corresponding to each step involved in the polymer encapsulation strategy in accordance with the present disclosure. The inset shows zoomed-in spectra highlighting the shifts in the LSPR wavelength.

FIG. 3B is an exemplary embodiment of an LSPR shift corresponding to each step involved in the polymer encapsulation strategy in accordance with the present disclosure. The error bars represent standard deviations from three different samples.

FIG. 3C is an exemplary embodiment of an LSPR shift upon exposure of polymer-encapsulated biosensors to different concentrations of anti-IgG before (black) and after SDS (red) treatment in accordance with the present disclosure. The error bars represent standard deviations from three different samples.

FIG. 3D is an exemplary embodiment of an LSPR wavelength shift after alternate exposure to anti-IgG and SDS in accordance with the present disclosure. The error bars represent standard deviations from three different samples.

FIG. 3E is an exemplary embodiment of the retained biorecognition capability of biosensors with and without polymer encapsulation over multiple capture/release cycles of the analyte in accordance with the present disclosure. The error bars represent standard deviations from three different samples.

FIG. 3F is an exemplary embodiment of the retained biorecognition capability of AuNR-IgG bioconjugates with and without polymer encapsulation stored at room temperature, 40 and 60° C. for different durations in accordance with the present disclosure. The error bars represent standard deviations from three different samples.

FIG. 4A is an exemplary embodiment of (%) the retained biorecognition capability of pristine and polymer-encapsulated AuNR-IgG-based biosensors after being subjected to different conditions of proteolytic degradation at room temperature in accordance with the present disclosure. The error bars represents the standard deviation from three independent samples.

FIG. 4B is an exemplary embodiment of an LSPR wavelength shift after alternate exposure of polymer-encapsulated biosensors to anti-IgG and NaOH in accordance with the present disclosure. The error bars represents the standard deviation from three independent samples.

FIG. 4C is an exemplary embodiment of an LSPR wavelength shift after alternate exposure of polymer-encapsulated biosensors to anti-IgG and PA in accordance with the present disclosure. The error bars represents the standard deviation from three independent samples.

FIG. 4D is an exemplary embodiment of an LSPR wavelength shift after alternate exposure of polymer-encapsulated biosensors to anti-IgG and glycine buffer in accordance with the present disclosure. The error bars represents the standard deviation from three independent samples.

FIG. 5A is an exemplary embodiment of a representative TEM image of Au nanorattles (AuNRT) used as nanotransducers in accordance with the present disclosure.

FIG. 5B is an exemplary embodiment of a normalized vis-NIR extinction spectra of AuNRT and AuNRT conjugated with a NGAL antibody in solution depicting ˜10 nm redshift in the LSPR wavelength in accordance with the present disclosure.

FIG. 5C is an exemplary embodiment of an LSPR shift upon exposure of AuNRT-NGAL antibody bioconjugates to different concentrations of NGAL before (black) and after SDS (red) treatment in accordance with the present disclosure. The error bar represents standard deviations from three different samples.

FIG. 5D are exemplary embodiments of normalized vis-NIR extinction spectra of AuNRT-NGAL antibody bioconjugates before and after exposure to 5 μg/ml NGAL depicting ˜20 nm redshift in LSPR wavelength in accordance with the present disclosure.

FIG. 5E are exemplary embodiments of normalized UV-vis extinction spectra of AuNRT-NGAL antibody corresponding to each step involved (left): immobilization of AuNRT-NGAL antibody bioconjugates on glass substrates; capture of NGAL on PEGylated AuNRT-NGAL antibody bioconjugates; exposure to SDS solution to remove NGAL; recapture of NGAL. Inset shows zoomed in spectra highlighting the shifts in the LSPR wavelength, and LSPR shifts corresponding to biodetection and sensor refreshing (right) in accordance with the present disclosure.

FIG. 5F is an exemplary embodiment of an LSPR wavelength shift after exposure of AuNRT-NGAL antibody bioconjugates to different concentrations of APTMS and TMPS monomers (top), retained biorecognition capability with increasing polymer thickness/monomer concentration (bottom left), retained biorecognition capability of AuNRT-NGAL antibody bioconjugates with increasing monomer concentration, after SDS treatment (bottom right) in accordance with the present disclosure. Error bars represent standard deviations from three different samples.

FIG. 5G is an exemplary embodiment of an extinction spectra corresponding to each step involved in the polymer encapsulation strategy of AuNRT-NGAL antibody bioconjugates in accordance with the present disclosure. The inset shows zoomed-in spectra highlighting the shifts in the LSPR wavelength.

FIG. 5H is an exemplary embodiment of an LSPR shift upon exposure of polymer-encapsulated AuNRT-NGAL antibody bioconjugates to different concentrations of NGAL before (black) and after SDS (red) treatment in accordance with the present disclosure. The error bar represents standard deviations from three different samples.

FIG. 5I is an exemplary embodiment of the retained biorecognition capability of biosensors with and without polymer encapsulation over multiple capture/release cycles of NGAL in accordance with the present disclosure. The error bar represents standard deviations from three different samples.

FIG. 5J is an exemplary embodiment of the retained biorecognition capability of AuNR-IgG bioconjugates with and without polymer encapsulation stored at room temperature, 40 and 60° C. for different durations in accordance with the present disclosure. The error bar represents standard deviations from three different samples.

FIG. 6 is an exemplary embodiment of a schematic illustration depicting the concept of MOF-based bioassay preservation in accordance with the present disclosure.

FIG. 7A is an exemplary embodiment of a schematic depicting ZIF-90 removal and assay procedure in accordance with the present disclosure.

FIG. 7B is an exemplary embodiment of AFM images for antigen-coated microtiter plate surface (i) with ZIF-90 and (ii) without ZIF-90 in accordance with the present disclosure.

FIG. 7C is an exemplary embodiment of an AFM scratch test on silicon exhibiting in accordance with the present disclosure.

FIG. 7D is an exemplary embodiment of a section analysis of the scratch test on silicon shown in FIG. 7C exhibiting that the average thickness of ZIF layer is 40±5 nm in accordance with the present disclosure.

FIG. 7E is an exemplary embodiment of SEM images for antigen-coated microtiter plate surface with ZIF-90 and (ii) without ZIF-90 in accordance with the present disclosure.

FIG. 7F is an exemplary embodiment of Raman spectra obtained from antigen-coated microtiter plate before and after growing ZIF-90 layer in accordance with the present disclosure.

FIG. 7G is an exemplary embodiment of XRD pattern obtained from ZIF-90 encapsulated antigens on silicon substrate and simulated ZIF-90 XRD patterns in accordance with the present disclosure.

FIG. 8 is an exemplary embodiment of ELISA standard curves obtained from freshly prepared microtiter plates coated with SARS-CoV-2 S1 protein and ZIF-90 treated microtiter plates coated with SARS-CoV-2 S1 in accordance with the present disclosure.

FIG. 9A is an exemplary embodiment of ELISA standard curves obtained from microtiter plates coated with SARS-CoV-2 S1 protein and stored under different conditions for 8 days in accordance with the present disclosure.

FIG. 9B is an exemplary embodiment of ELISA standard curves obtained from microtiter plates coated with SARS-CoV-2 S1 protein and stored under different conditions for 24 days in accordance with the present disclosure.

FIG. 9C is an exemplary embodiment of ELISA standard curves obtained from microtiter plates coated with SARS-CoV-2 S1 protein and stored under different conditions for 32 days in accordance with the present disclosure.

FIG. 9D is an exemplary embodiment of preservation efficacy as calculated from the OD values in the linear range of ELISA standard curves of SARS-CoV-2 S1 protein-coated microtiter plates in accordance with the present disclosure.

FIG. 9E is an exemplary embodiment of a comparison of LODs of SARS-CoV-2 S1 protein coated plates stored under different conditions in accordance with the present disclosure.

FIG. 9F is an exemplary embodiment of OD values obtained from SARS-CoV-2 S1 protein-coated plates after treatment with different concentrations of proteases in accordance with the present disclosure.

FIG. 9G is an exemplary embodiment of preservation efficacy of ZIF-90 protected plates after thermal treatment and then exposing to protease in accordance with the present disclosure.

FIG. 9H is an exemplary embodiment of a comparison of LODs of SARS-CoV-2 N protein coated microtiter plates stored under different conditions in accordance with the present disclosure.

FIG. 9I is an exemplary embodiment of preservation efficacy as calculated from the OD values in the linear range of ELISA standard curves of SARS-CoV-2 N protein pre-coated plates in accordance with the present disclosure.

FIG. 10A is an exemplary embodiment of a heat map of preservation efficacy of surface-bound S1 protein and stored for different duration with ZIF-90 and without ZIF-90in accordance with the present disclosure.

FIG. 10B is an exemplary embodiment of a heat map of preservation efficacy of surface-bound N protein stored for different duration with ZIF-90 and without ZIF-90 in accordance with the present disclosure.

FIG. 11A is an exemplary embodiment of ZIF-90 encapsulation procedures of COVID-19 patient samples in accordance with the present disclosure.

FIG. 11B is an exemplary embodiment of an SEM image of pristine paper substrate (top) and after drying plasma encapsulating ZIF-90 crystals on the surface (bottom) in accordance with the present disclosure.

FIG. 11C is an exemplary embodiment of Raman spectra (top) obtained from pristine paper substrate and after drying plasma encapsulating ZIF-90 crystals on the surface, and XRD patterns (bottom) of ZIF-90 encapsulated patient sample and simulated ZIF-90 pattern in accordance with the present disclosure.

FIG. 11D is an exemplary embodiment of preservation efficacy of plasma samples from COVID-19 patients with and without ZIF-90 encapsulation after storage at 40° C. for 3 weeks in accordance with the present disclosure.

### DETAILED DESCRIPTION OF THE DISCLOSURE

### Refreshable Nanobiosensor Based on Organosilica Encapsulation of Biorecognition Elements

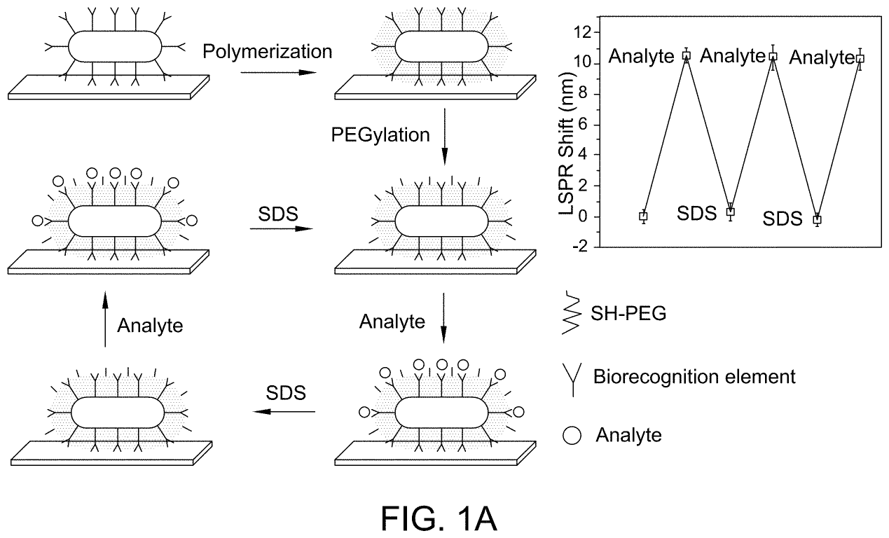

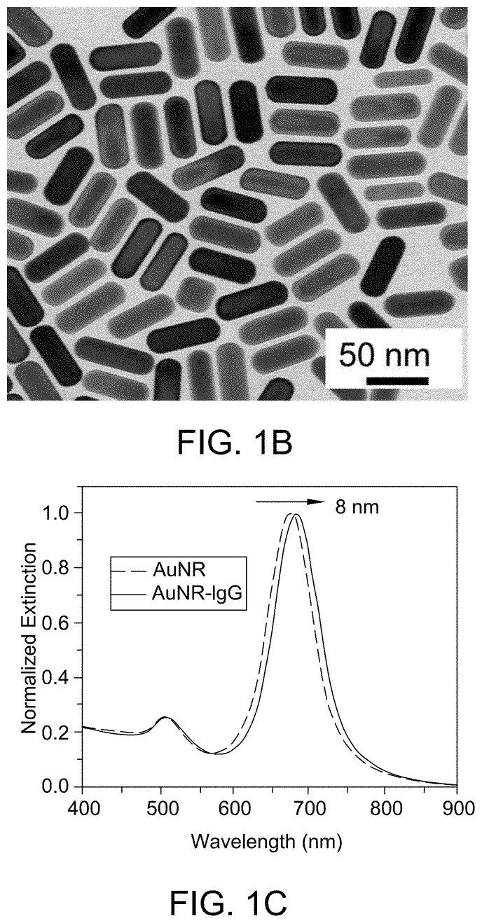

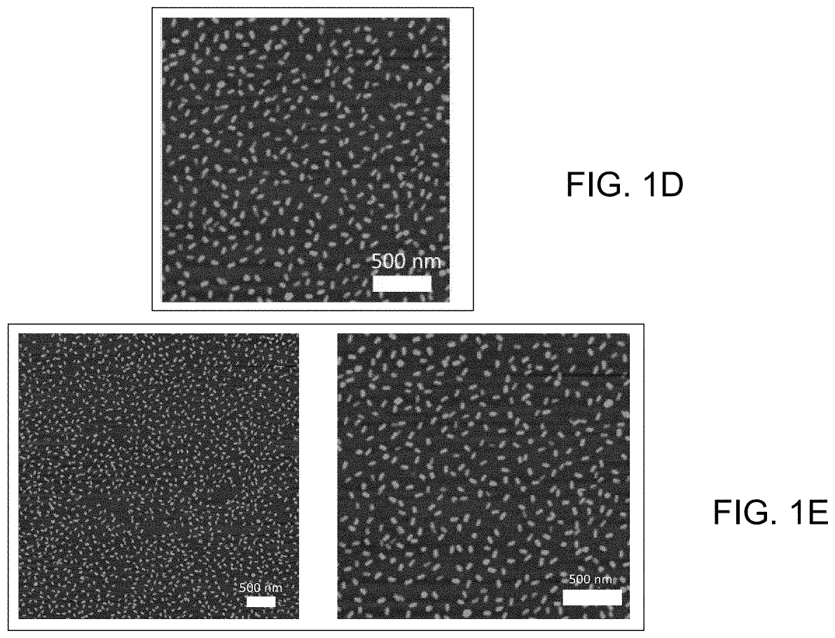

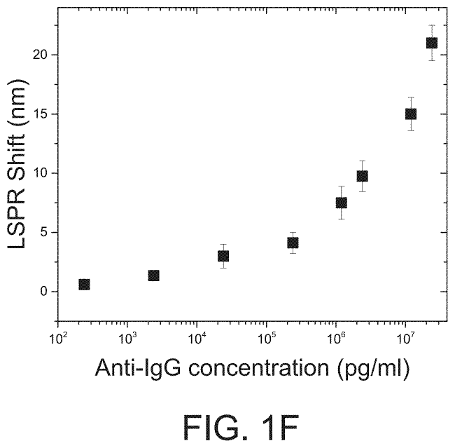

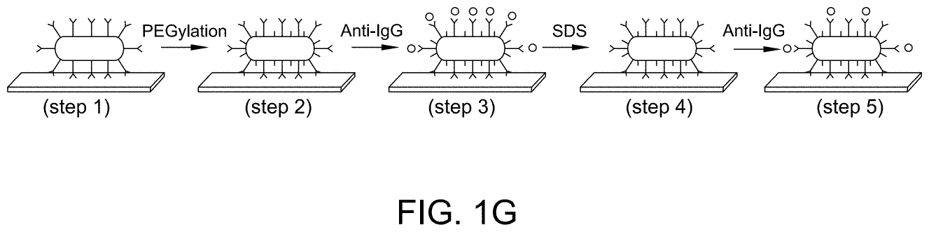

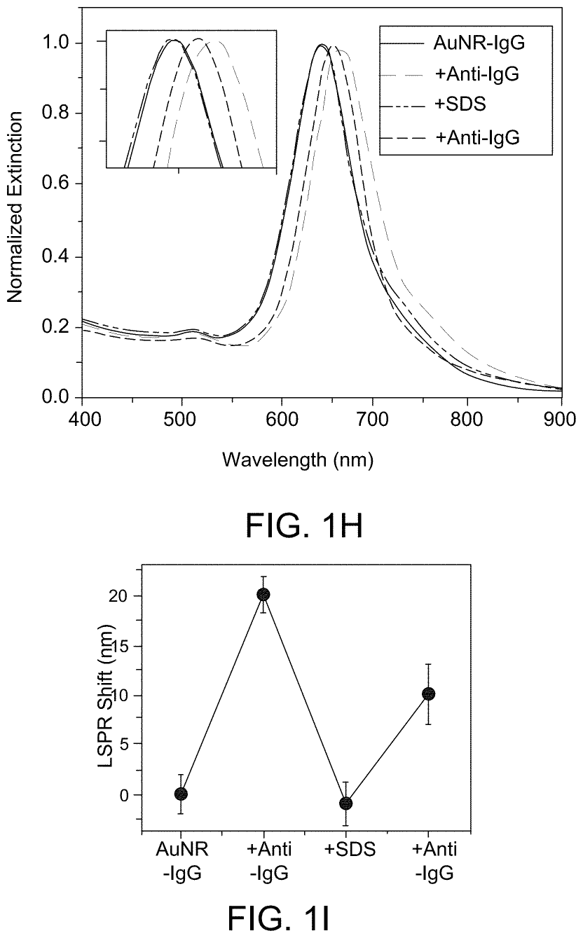

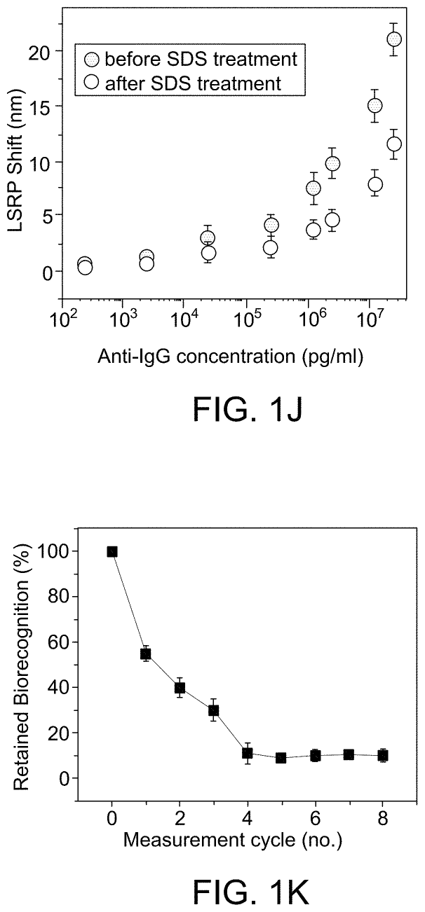

Implantable and wearable biosensors that enable monitoring of biophysical and biochemical parameters over long durations are highly attractive for early and pre-symptomatic diagnosis of pathological conditions and timely clinical intervention. Poor stability of antibodies used as biorecognition elements and the lack of effective methods to refresh the biosensors upon demand without severely compromising the functionality of the biosensor remain significant challenges in realizing protein biosensors for long-term monitoring. Herein, a novel method is disclosed (FIG. 1A) involving organosilica encapsulation of antibodies for preserving their biorecognition capability under harsh conditions, typically encountered during the sensor refreshing process, and elevated temperature. Specifically, a simple aqueous rinsing step using sodium dodecyl sulfate (SDS) solution refreshes the biosensor by dissociating the antibody-antigen interactions. Encapsulation of the antibodies with an organosilica layer is shown to preserve the biorecognition capability of otherwise unstable antibodies during the SDS treatment, thus ultimately facilitating the refreshability of the biosensor over multiple cycles. The methods disclosed herein demonstrate the refreshability of plasmonic biosensors for anti-IgG (model bioanalyte) and neutrophil gelatinase-associated lipocalin (NGAL) (a biomarker for acute and chronic kidney injury). The novel encapsulation approach demonstrated is easily extended to other transduction platforms to realize refreshable biosensors for monitoring of protein biomarkers over long durations.

As demonstrated herein, the encapsulation of antibodies immobilized on plasmonic nanostructures with an organosilica layer renders refreshability to the biosensors by preserving the antibody biorecognition ability upon subjecting them to harsh chemical treatment for dissociating the antibody-antigen interactions. This novel method overcomes the challenges associated with poor stability of immobilized antibodies under harsh conditions and opens up opportunities for realizing wearable and implantable biosensors for continuous monitoring of protein biomarkers over long durations.

### RESULTS AND DISCUSSION

AuNR-IgG-Based Biosensor. Rabbit IgG and goat anti-rabbit IgG were employed as a model of the antibody-antigen pair and gold nanorods (AuNRs) as plasmonic nanotransducers. AuNRs were synthesized using a seed-mediated method. Transmission electron micrograph (TEM) images revealed the length and diameter of the AuNRs as 57.5±2.1 and 19.7±2.6 nm, respectively (FIG. 1B). The antibodies were conjugated to a bifunctional poly(ethylene glycol) (COOH-PEG-SH) chain to obtain IgG-PEG-SH. Subsequently, IgG-PEG-SH was anchored to the AuNR surface via an Au—S linkage. Poly(ethylene glycol) (PEG) chains offer two important advantages: (i) increased accessibility of IgG to target biomolecules by acting as a flexible linker and (ii) minimization of nonspecific binding owing to their high hydrophilicity. The immobilization of IgG-PEG-SH on AuNRs in solution resulted in an ˜8 nm red shift in the longitudinal LSPR wavelength of AuNRs, corresponding to an increase in the refractive index of the medium surrounding AuNRs (FIG. 1C). To realize plasmonic biosensors, the AuNR-IgG bioconjugates were uniformly adsorbed onto the 3-mercaptopropyl-trimethoxysi-lane (MPTMS)-functionalized glass substrates. Atomic force microscopy (AFM) images of the modified glass substrates revealed uniform distribution of AuNR-IgG bioconjugates with no signs of aggregation or patchiness (FIGS. 1D and 1E). To minimize nonspecific binding, glass substrates with AuNR-IgG bioconjugates were exposed to thiol-terminated poly(ethylene glycol) (SH-PEG), which is expected to graft to the exposed regions of AuNRs and serve as a blocking layer. To investigate their biosensing performance, these plasmonic biochips were exposed to different concentrations of anti-IgG, which binds specifically to the IgG immobilized on the AuNRs. The LSPR wavelength of AuNR exhibited a monotonic red shift with an increase in the concentration of anti-IgG (FIG. 1F). The limit of detection (LOD defined as: mean +3 σ of the blank) of these biochips was found to be 240 pg/mL.

Refreshability of the AuNR-IgG Biosensor. As mentioned above, antigens (anti-IgG in this case) are recognized and captured by antibodies (IgG conjugated to AuNRs in the present case) through noncovalent interactions, which are disrupted by subjecting them to extreme pH or high concentrations of salt or surfactants. Sodium dodecyl sulfate (SDS), an anionic surfactant frequently used in cleaning and hygiene products (e.g., toothpaste and mouthwash), was used as a chemical agent to disrupt the antigen-antibody interactions. SDS, known to disrupt protein conformation, binds relatively uniformly along the protein chain with its hydrophobic tail conferring net charge on proteins and thus exposing (unfolding) the otherwise buried regions of the protein. Exposure of the bound antibody-antigen pair to SDS results in electrostatic repulsion between the antibody and antigen, thus overcoming the noncovalent interactions between them. Dissociation of the antibody-antigen pair essentially refreshes the binding sites and enables the reuse of the biosensor. The refractive index sensitivity of the AuNRs monitors each step along this process (FIG. 1G). Extinction spectra (FIG. 1H) and the LSPR wavelength of AuNR (FIG. 1I) were obtained following each step in the procedure: immobilization of AuNR-IgG bioconjugates on glass substrates (step 1); PEGylation of AuNR-IgG bioconjugates to minimize nonspecific binding (step 2); binding of anti-IgG to IgG (step 3); exposure to SDS solution (step 4); and rebinding of anti-IgG to IgG (step 5).

The LSPR wavelength of AuNR exhibited a ˜20 nm red shift after exposure to 24 μg/mL anti-IgG (FIGS. 1H, 1I). After exposure to SDS, a ˜21 nm blue shift was observed suggesting complete removal of anti-IgG and thus leaving behind AuNR-IgG bioconjugates on the plasmonic biochip, ready for another cycle of antigen detection (FIGS. 1H,1I). However, when SDS-treated plasmonic biochips were exposed to the same concentration of anti-IgG, the biochip lost ˜50% sensitivity as evident from only a ˜10 nm red shift as opposed to ˜20 nm observed in the pristine biochip (FIGS. 1H,1I). The nearly 50% loss in biorecognition capability after SDS treatment was consistent over a broad range of anti-IgG concentration (FIG. 1J). This is not surprising because in the process of dissociating anti-IgG:IgG complex, SDS partially denatures the immobilized IgG and thus results in the loss of its biorecognition capability. Consequently, with every cycle of SDS washing, a progressive degradation in the biorecognition capability was noted and by fourth cycle the plasmonic biochips exhibited only ˜10% sensitivity compared to that in the pristine condition (FIG. 1K). Thus, this challenge of antibody denaturation and subsequent loss in biorecognition capability needs to be overcome to realize the refreshable sensor. Toward this goal, antibody encapsulation was explored as a strategy to render protection against harsh and potentially denaturing conditions and ultimately leads to refreshable biosensors.

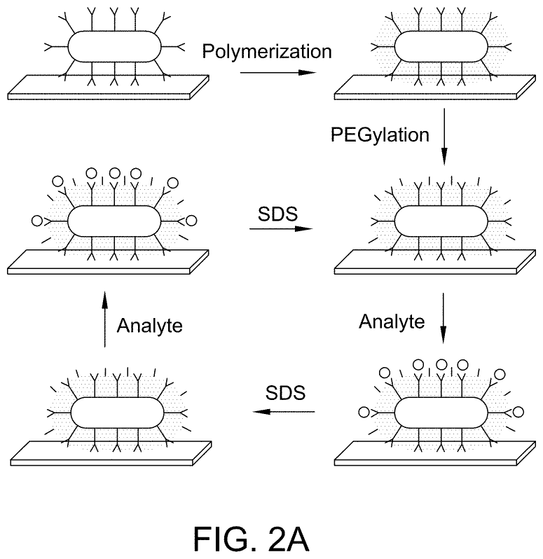

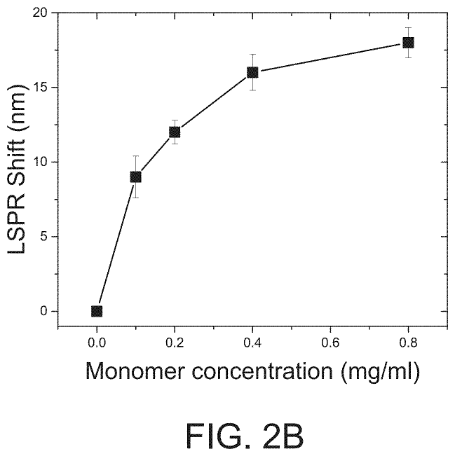

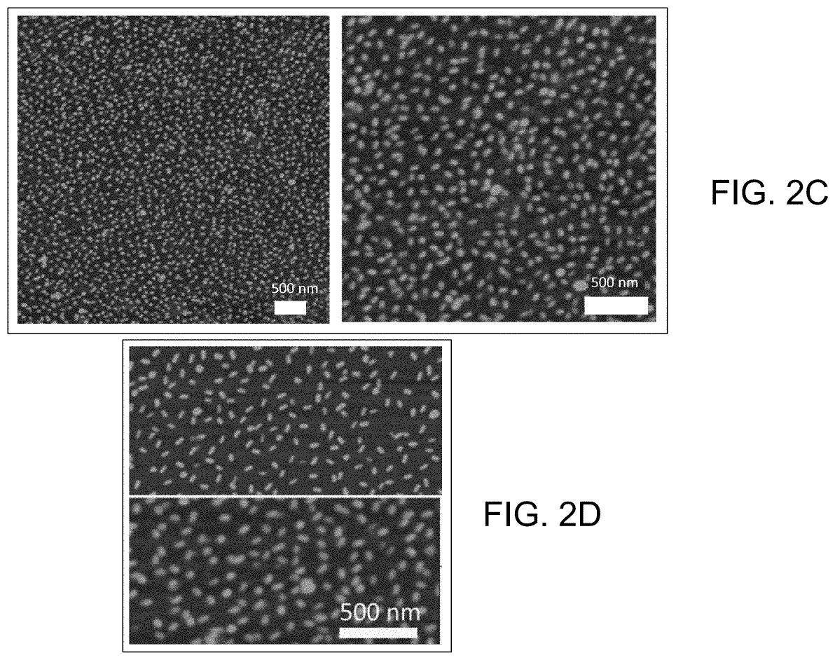

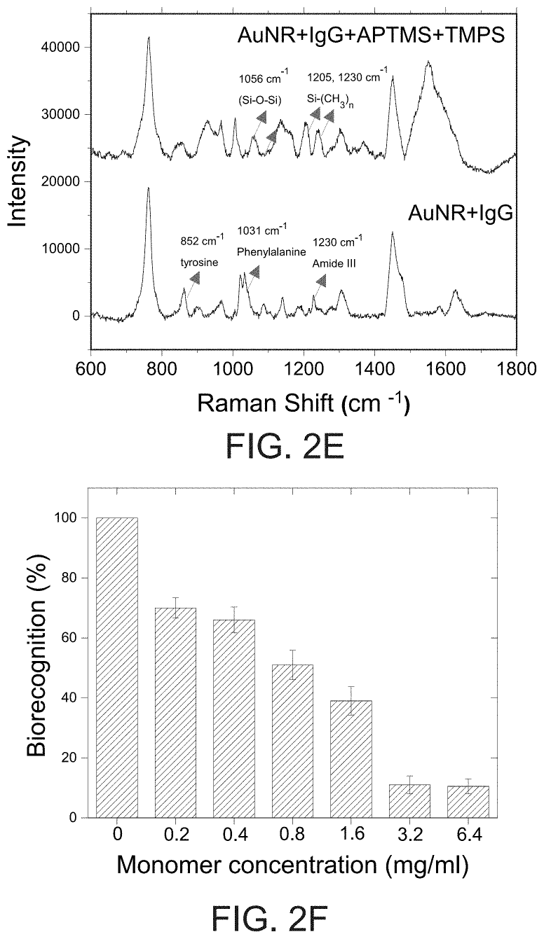

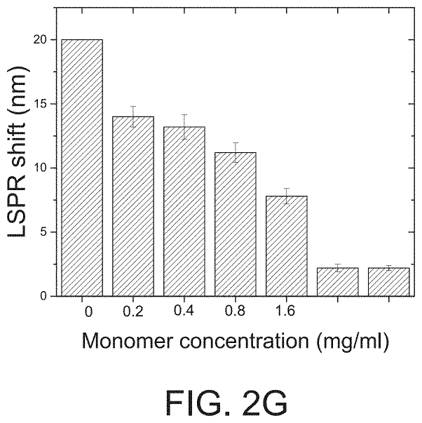

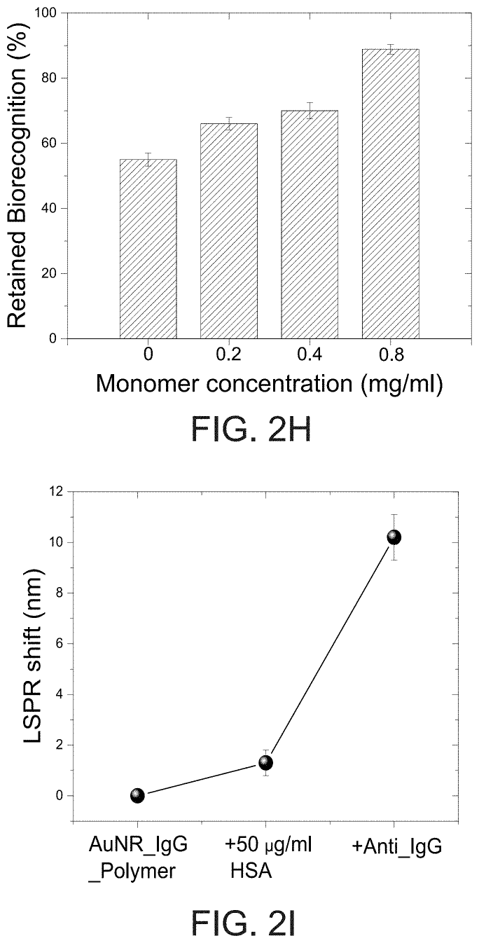

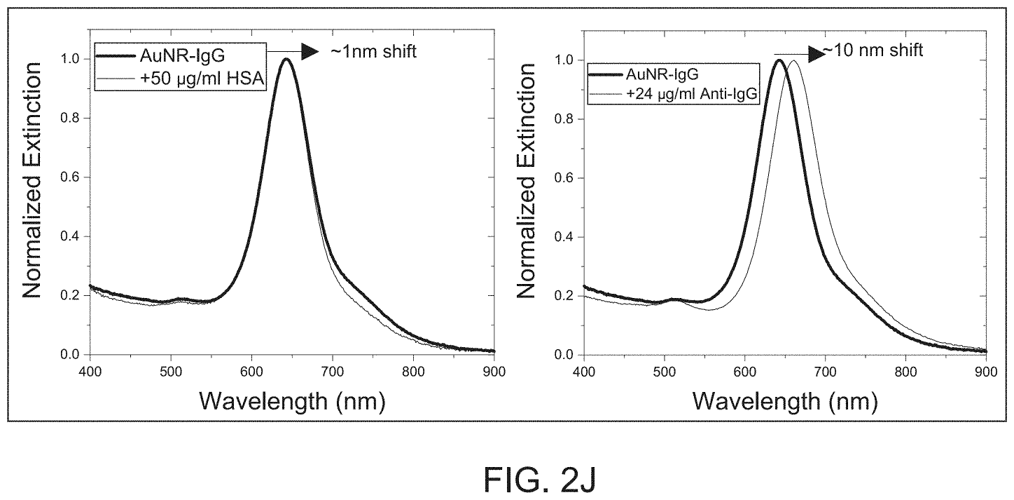

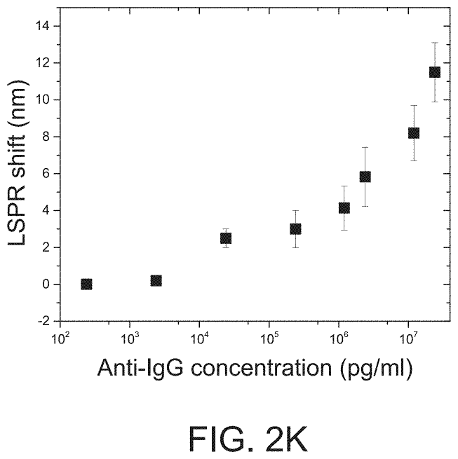

Polymer Encapsulation Strategy to Achieve Refreshability. Previously, an in situ polymerization technique was demonstrated for preserving the activity (biopreservation) of an enzyme, immobilized on plasmonic nanostructures, subjected to harsh conditions such as proteases and high temperature. Similarly, encapsulation of immobilized antibodies with an in situ formed polymer layer preserves its biorecognition capability against SDS washing (FIG. 2A). Following the immobilization of AuNR-IgG bioconjugates on glass substrates, a polymer encapsulation layer is formed through copolymerization of (3-aminopropyl) trimethoxysilane (APTMS) and trimethoxy(propyl)silane (TMPS) on AuNR and around immobilized IgG. The methoxy group of TMPS and APTMS undergoes rapid hydrolysis to form methanol and trisilanols. Hydrolysis is followed by condensation of the silanols, which results in the formation of an amorphous aminopropyl functional polymer layer consisting of Si—O—Si bonds and functional end groups such as hydroxyl (—OH), amine (—NH3+), and methyl (—CH3). These end groups interact noncovalently via hydrogen bonding, hydrophobic, and electrostatic interactions with AuNR-IgG bioconjugates resulting in the formation of a stable organosilica layer around them. Next, bifunctional PEG (methoxy-PEG-silane) was covalently grafted on to the free regions of the organosilica layer. The methoxysilane group of PEG undergoes hydrolysis followed by condensation with the reactive silanol group present on the polymer surface, resulting in the formation of a stable covalent siloxane bond (Si—O—Si). PEG chains shield the functional groups present on the polymer layer, thus minimizing nonspecific binding. Biosensors with the organosilica protective layer were then exposed to a range of concentration of anti-IgG (240 pg to 24 μg) to allow antigen-antibody binding. The plasmonic biochips were subsequently exposed to an aqueous solution of SDS to overcome the noncovalent interactions, dissociate the antibody-antigen pair, and refresh the biochip. The restored biosensor was repeatedly exposed to anti-IgG to assess the refreshability of the biosensor.

Characterization and Optimization of the Organosilica Polymer Layer Thickness. Formation of the polymer encapsulation layer on AuNR-IgG bioconjugates was confirmed by redshift in the LSPR wavelength (FIG. 2B) and AFM imaging (FIG. 2C). The AFM image of the polymer-encapsulated bioconjugates revealed a change in the morphology corresponding to the formation of the organosilica polymer layer (FIGS. 2D, 1E and 2C). The presence of an organosilica polymer layer on the AuNR-IgG bioconjugates was further confirmed by surface-enhanced Raman scattering (SERS) spectroscopy (FIG. 2E). Pristine AuNR-IgG bioconjugates exhibited Raman bands at 852, 1031, 1230, and 1620-40 cm-1 corresponding to tyrosine, phenylalanine, amide III, and amide I of IgG. After the formation of an organosilica layer, Raman bands were observed at 1024, 1056, 1205, and 1230 cm-1 corresponding to Si—O—R stretching, Si—O—Si stretching, and —CH2 bending.

It is important to note that if the entire antibody, including its antigen binding sites, is encapsulated within the polymer layer, it will severely compromise the biorecognition capability of IgG. On the other hand, if the encapsulation is insufficient, then the protection against SDS and long-term stability of the antibody will be limited. Therefore, the thickness of the encapsulating polymer layer is critical to provide both access for analyte binding and protection against harsh conditions. The thickness of the organosilica layer is controlled either by varying the polymerization time or by changing the concentration of the APTMS and TMPS monomers. The concentration of monomers was varied while keeping the polymerization time constant (10 min), as it offers better control and repeatability over multiple batches. The red shift in the LSPR wavelength of the AuNR corresponding to the formation of the organosilica layer increased with an increase in the concentration of monomers, indicating a gradual increase in the thickness of the polymer layer (FIG. 2B). Pristine plasmonic biochips with no polymer encapsulation, which corresponds to the maximum availability of antibody binding sites, displayed a ˜20 nm red shift (treated as 100% biorecognition capability). As the thickness of the polymer layer was gradually increased, the plasmonic biochips exhibited a progressive decrease in biorecognition capability (FIGS. 2F and 2G). Here, biorecognition capability is defined as the percentage of the red shift upon specific binding of anti-IgG to IgG after encapsulation with a polymer layer compared with the red shift obtained from the same batch of biochips before encapsulation. An increase in the thickness of the polymer layer rendered biosensors increasingly stable against SDS washing, thus enabling their reusability. The percentage of retained biorecognition capability was used to quantitatively evaluate the preservation efficacy of the polymer encapsulation strategy. It was calculated as the percentage of the red shift upon specific binding of goat anti-rabbit IgG to the rabbit IgG on a restored biochip after one or more cycles of SDS treatment compared with the red shift obtained from the same batch of the biosensor before SDS treatment. For example, samples with no polymer encapsulation and before SDS treatment exhibited ˜20 nm red shift, showing 100% biorecognition capability (FIG. 2F), and after SDS treatment displayed ˜10 nm red shift corresponding to only 50% retained biorecognition capability (FIG. 2H). On the other hand, plasmonic biochips with polymer encapsulation corresponding to the monomer concentration of 0.8 mg/mL exhibited a red shift of 11.5 nm before SDS treatment and red shift of 10.5 nm following SDS treatment, corresponding to ˜90% retained biorecognition capability (FIG. 2H). This significant improvement in the biorecognition capability against SDS underscores the importance of polymer encapsulation of the antibody for the successful refreshability of the biosensors. By gradually changing the monomer concentration/thickness of the polymer layer, a balance was found between the loss of biorecognition capability and an increase in the preservation efficacy of polymer encapsulation of AuNR-IgG conjugates to achieve refreshability. Considering the retained biorecognition capability of ˜90% for the monomer concentration of 0.8 mg/mL, this condition was employed in subsequent experiments.

Specificity of the Polymer-Encapsulated AuNR-IgG Biosensor. To determine the specificity of bioconjugates after polymer encapsulation, shifts in the LSPR wavelength of AuNR were measured after the exposure of plasmonic biochips to high concentration (50 μg/mL) of interfering protein such as human serum albumin (HSA) (FIGS. 2I and 2J). The LSPR shift corresponding to the exposure of the polymerized biosensor to 50 μg/mL HSA was only ˜1 nm, which is significantly lower than the ˜10.5 nm red shift obtained upon exposure to 24 μg/mL anti-IgG. This low nonspecific binding was attributed to the covalently grafted PEG chains on the free surface of the organosilica polymer layer, which are known to resist nonspecific protein adsorption. Further, the sensing capability of the polymer-encapsulated plasmonic biosensors was probed by exposing them to different concentrations of anti-IgG and monitoring the LSPR shift of the AuNR. As expected, a monotonic increase was observed in the LSPR wavelength with an increase in the anti-IgG concentration (FIG. 2K). The limit of detection (defined as: mean +3 σ of the blank) of these biochips was found to be 3.7 ng/mL.

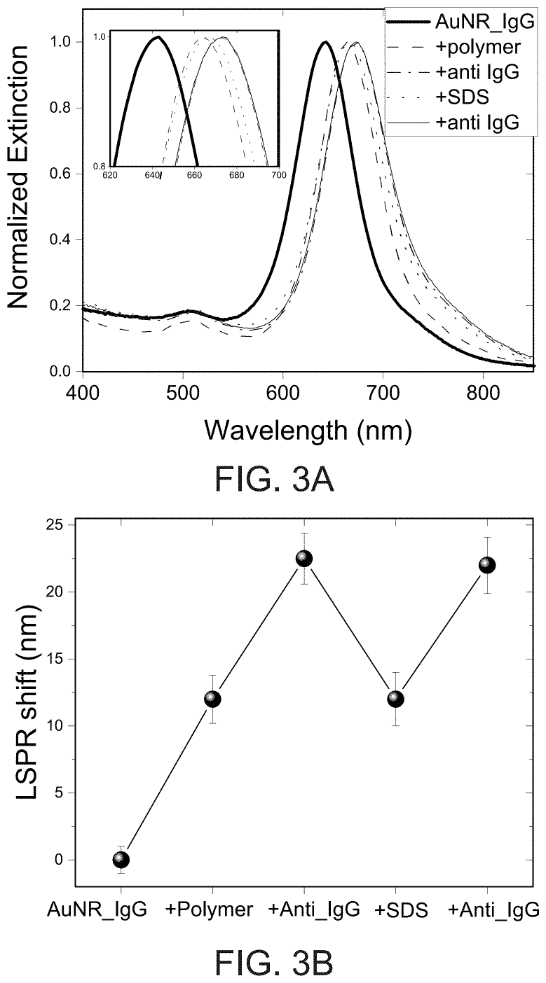

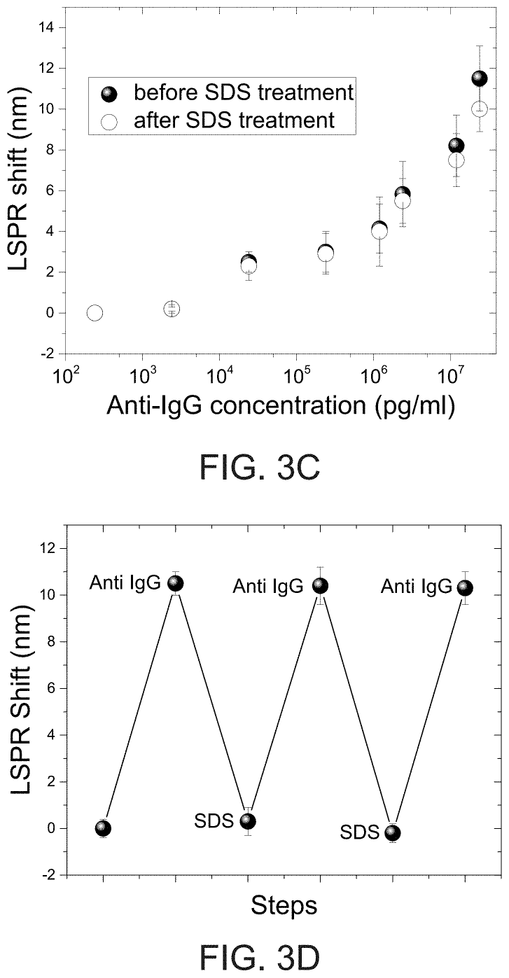

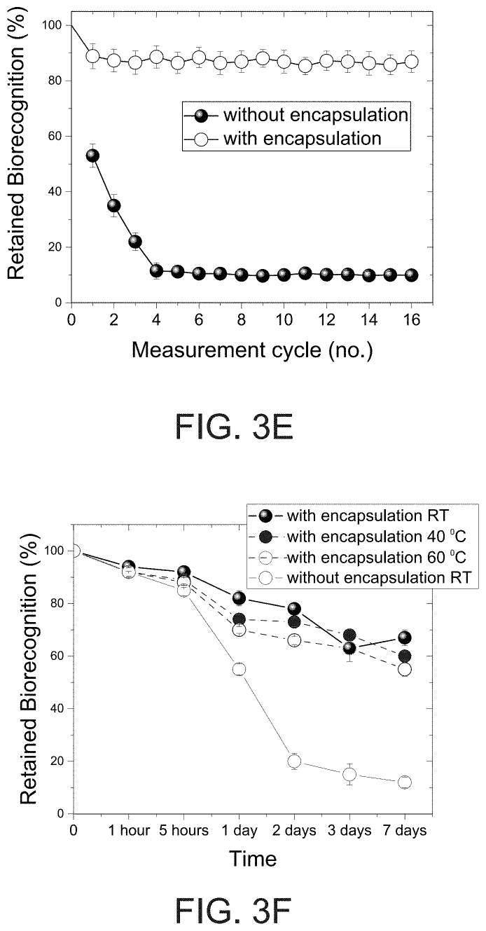

Refreshability of the Polymer-Encapsulated AuNR-IgG Biosensor. Next, the refreshability of the polymer-encapsulated biosensors was investigated. FIG. 3A shows the extinction spectra obtained after: immobilization of AuNR-IgG; formation of the organosilica layer; specific binding of anti-IgG (24 μg/mL) to IgG, which resulted in a ˜10.5 nm red shift; refreshing the plasmonic biochip by SDS washing, which resulted in ˜10.5 nm blue shift suggesting the dissociation of the anti-IgG:IgG pair; reuse of the refreshed biosensor by exposing it again to 24 μg/mL anti-IgG resulting in a ˜10 nm red shift, thus depicting the refreshability of biosensors. All of the biosensors after polymerization and prior to exposure to the analyte were PEGylated. The PEGylation step after polymer encapsulation resulted in a small (˜0.5 nm) red shift, which is not shown in FIG. 3A. FIG. 3B depicts sequential LSPR shifts obtained following each of the aforementioned steps suggesting that the polymer encapsulation strategy provides the ability to reuse the biosensors without significantly compromising the biorecognition capability. Similar results were observed for different concentrations of anti-IgG suggesting stability and refreshability of the biosensors over a large range of concentrations (FIG. 3C).

The reusability of polymer-encapsulated plasmonic biochips was investigated over multiple cycles by subjecting the plasmonic biochips to repeated cycles of capture (exposure to anti-IgG) and release (exposure to SDS). Anti-IgG captured by the polymer-encapsulated AuNR-IgG conjugates was completely released with SDS treatment as confirmed by the LSPR blue shift, identical to the red shift observed during capture (FIG. 3D). The refreshed biosensor was exposed to a fresh batch of anti-IgG (24 μg/mL) each time, resulting in a ˜10 nm red shift suggesting the near-complete preservation of biorecognition capability of bioconjugates. The polymer-encapsulated AuNR-IgG preserved ˜80% of biorecognition capability even after 16 cycles of SDS treatment (FIG. 3E). On the other hand, biosensors without polymer encapsulation exhibited <40% of biorecognition capability after the second cycle and ˜10% after the fourth cycle (FIG. 3E). These results underscore the importance of a polymer encapsulation strategy to achieve refreshability without significantly compromising the biorecognition capability.

Long-Term Usability of the Polymer-Encapsulated AuNR-IgG Biosensor. Another important aspect of deploying a refreshable biosensor over a long duration of time is the long-term stability of the biorecognition element under ambient and even harsh conditions. Therefore, the efficacy of polymer encapsulation was tested to preserve the biorecognition capability of AuNR-IgG bioconjugates against harsh conditions that, without polymer encapsulation, would lead to protein denaturation and consequent loss in biorecognition capability. The plasmonic biosensors with and without polymer encapsulation were stored at room temperature, 40 and 60° C. for different times (1 and 5 h and 1, 2, 3, and 7 days) to monitor the changes in the biorecognition capabilities of the antibodies (FIG. 3F). After storage, plasmonic biochips were exposed to anti-IgG (24 μg/mL). Biochips with polymer encapsulation exhibited ˜70% retention of biorecognition capability after storage at room temperature (25° C.) for 1 week compared to an almost complete loss in biorecognition capability for biochips without polymer encapsulation. Significantly, the biochips with polymer encapsulation retained ˜60% of biorecognition capability even after storage at higher temperatures (40 and 60° C.) for a week. In contrast, pristine biochips lost more than 50% of biorecognition capability within 1 day and ˜90% after 1 week. The remarkable stability of the polymer-encapsulated AuNR-IgG bioconjugates possibly stems from the restricted mobility of the biomolecules, and thus impeding protein denaturation even under extreme conditions. That is, the noncovalent interactions between bioconjugates and organosilica layer impose steric hindrance on the antibodies, and thus restricting them from undergoing changes in secondary and tertiary structures (unfold).

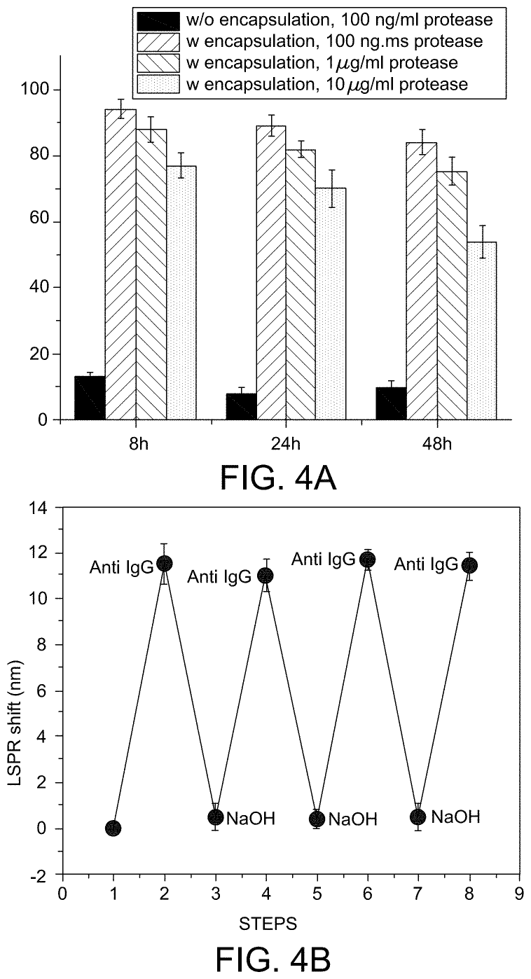

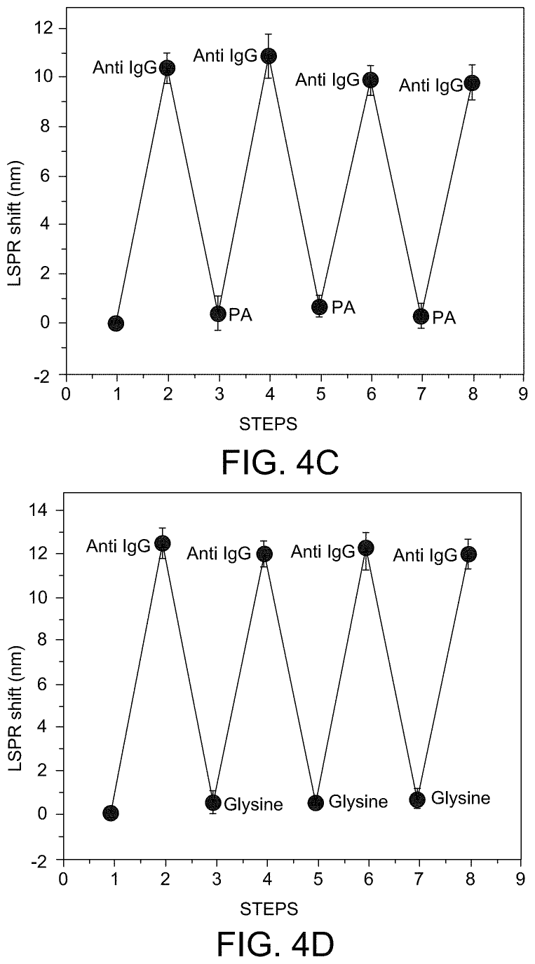

Biological Stability of the AuNR-IgG Biosensor. In addition to the remarkable thermal stability, which allows long-term usability, biosensors are required to be stable against biological agents such as proteases in patient serum/urine samples, which lead to proteolytic degradation of antibodies. Therefore, to probe the biological stability of polymer-encapsulated biosensors, AuNR-IgG based pristine and organosilica-stabilized biosensors were subjected to different concentrations of protease dissolved in synthetic urine for different time periods at room temperature. The biorecognition capability AuNR-IgG bioconjugates decreased to ˜8% for all conditions, while the polymer-encapsulated bioconjugates retained ˜90, ˜83, and ˜70% of the biorecognition capability when subjected to 100 ng/mL, 1μg/mL, and 10 μg/mL for 24 h, respectively (FIG. 4A). These results suggest that the organosilica layer significantly lowers the accessibility of the immobilized antibody to the protease, rendering excellent biological stability against proteolytic digestion.

Compatibility of the Encapsulation Strategy with Other Chemical Regeneration Agents. To further ascertain the universality of the polymer encapsulation strategy, the compatibility of the polymer encapsulation was explored with different regeneration techniques. Multiple capture/release cycles were performed using the following chemical regeneration agents: (i) acid-mediated regeneration using 0.1 M phosphoric acid (PA) solution; (ii) base-mediated regeneration using 50 mM sodium hydroxide (NaOH); and (iii) 10 mM glycine/HCl buffer (pH 2.8). These chemical regeneration approaches were investigated by subjecting the polymer-encapsulated plasmonic biochips to repeated cycles of capture (exposure to the analyte) and release (10 min exposure to aforementioned regeneration agents). FIGS. 4B, 4C, and 4D depict the LSPR wavelength shift obtained after alternate exposures of the analyte and the regeneration agent. The polymer-encapsulated biosensors exhibited ˜85% of biorecognition capability after treatment with different regeneration agents, including SDS, even after multiple wash cycles. Pristine plasmonic biosensors (i.e. unencapsulated) were also exposed to these regeneration agents. As expected, these biosensors exhibited a significant loss in biorecognition capability after treatment with the aforementioned regeneration agents. These results underscore the universality of polymer encapsulation of the immobilized antibodies rendering stability against various regeneration agents for achieving refreshability.

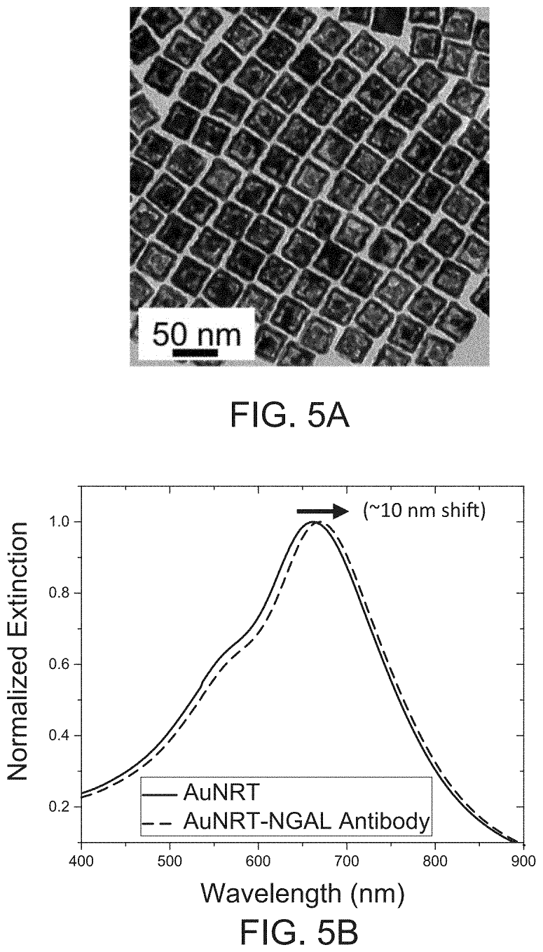

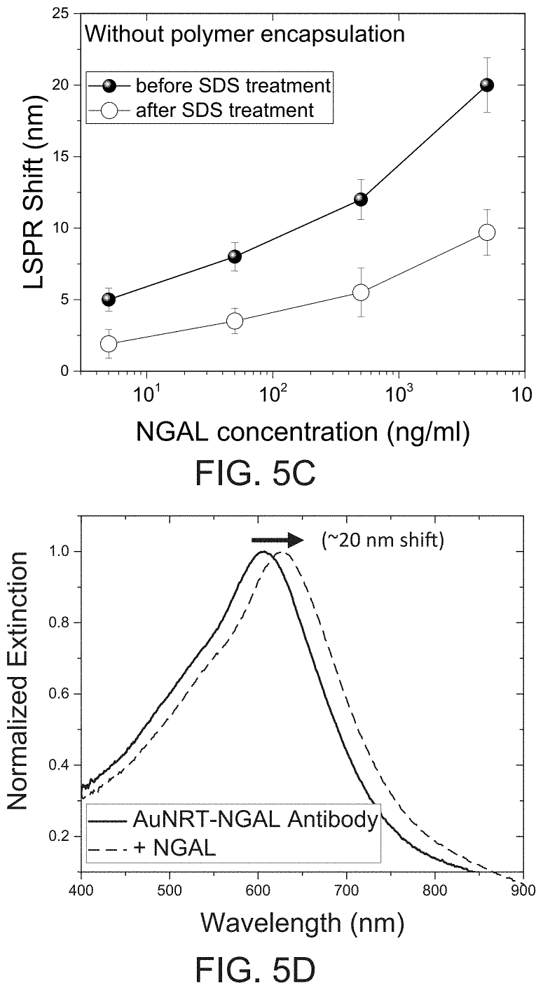

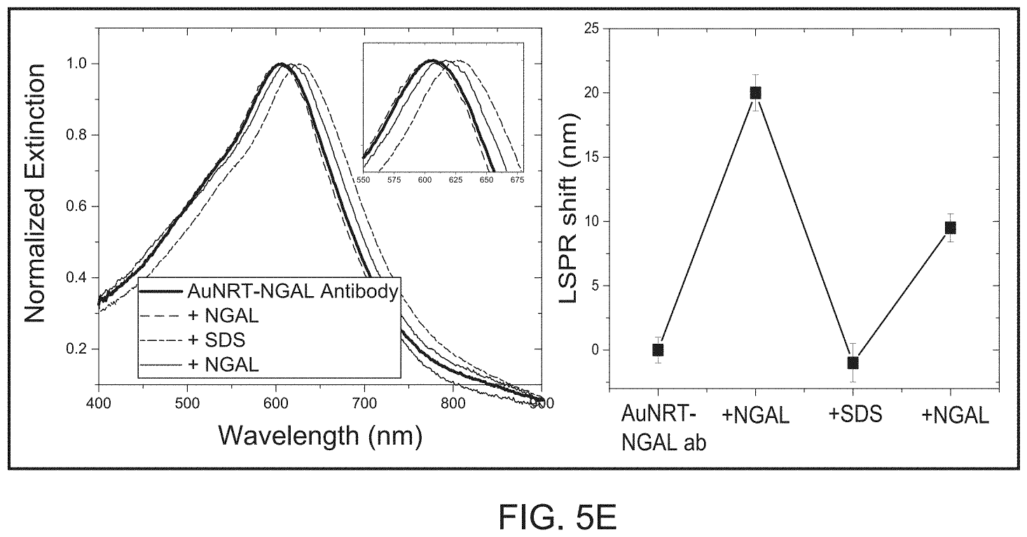

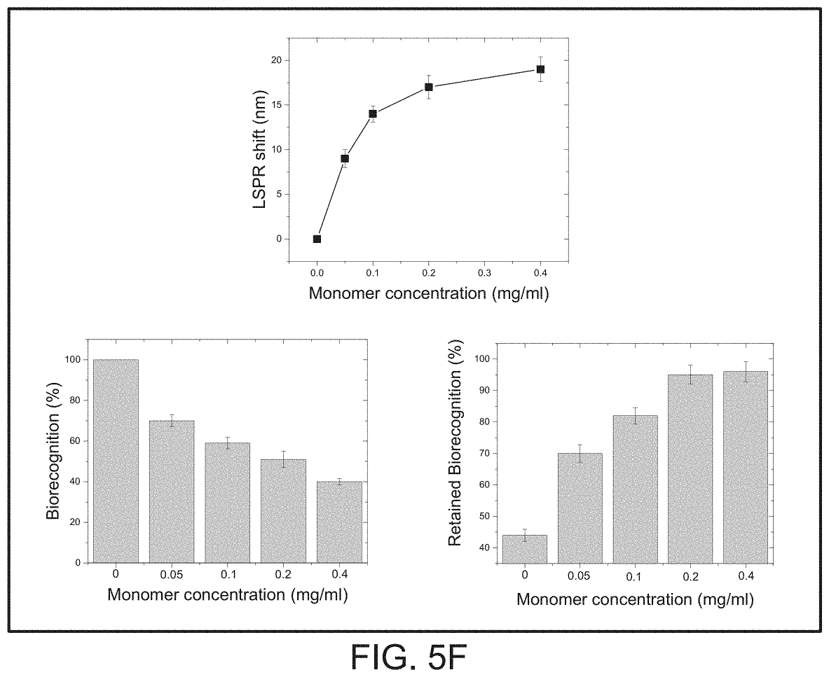

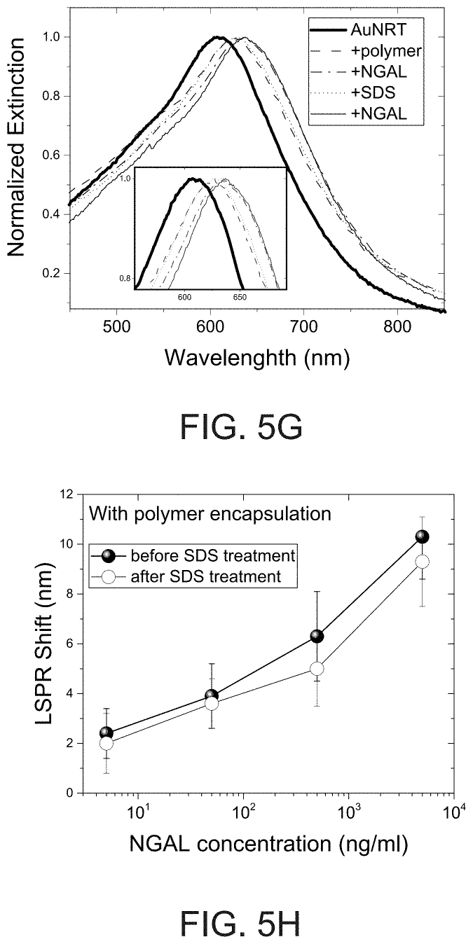

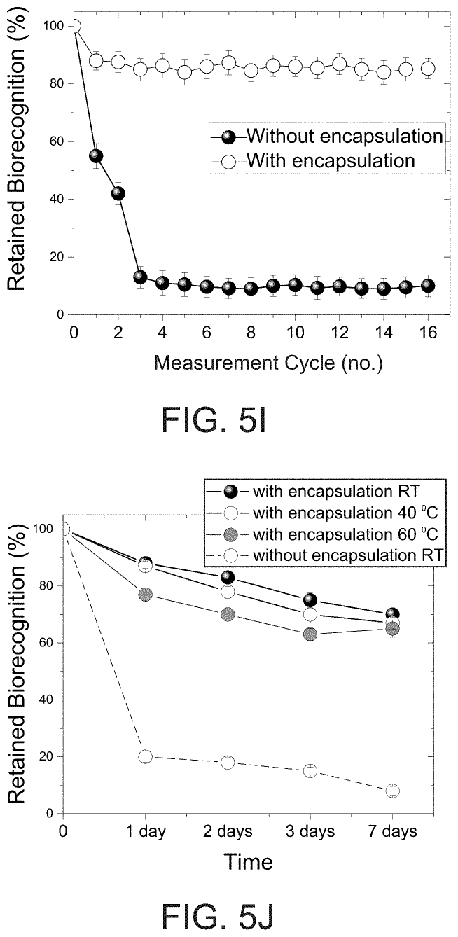

Universality of the Polymer Encapsulation Strategy. Finally, to verify the generality of the polymer encapsulation strategy for achieving the refreshable biosensor, gold nanorattles (AuNRT) were employed as plasmonic nanotransducers and neutrophil gelatinase-associated lipocalin (NGAL), a urinary biomarker for acute and chronic kidney injury, as target analytes. FIG. 5A shows the TEM image of AuNRTs with an edge length of 34.2±1.3 nm. Similar to IgG, anti-NGAL was conjugated to AuNRT and the conjugation was confirmed by a ˜10 nm red shift in the LSPR wavelength of AuNRT (FIG. 5B). Subsequently, AuNRT-anti-NGAL bioconjugates were immobilized on MPTMS-functionalized glass substrates. After PEGylation, the plasmonic biochips were exposed to NGAL (5 μg/mL) resulting in a ˜20 nm red shift in the LSPR wavelength of AuNRT (FIGS. 5C and 5D). To determine the refreshability, the biochips were treated with SDS to release the captured NGAL from AuNRT-anti-NGAL bioconjugates. The complete removal of NGAL was evidenced by a ˜20 nm blue shift in the LSPR wavelength. However, SDS treatment resulted in a ˜56% loss in biorecognition capability. Following the SDS treatment, AuNRT exhibited the LSPR shift of only ˜9.5 nm upon exposure to NGAL (5 μg/mL), as opposed to ˜20 nm observed before the SDS treatment (FIG. 5E). This loss in the biorecognition capability was observed over a broad range of NGAL concentrations, which is consistent with that observed in the case of AuNR-IgG bioconjugates (FIG. 5C). To overcome this loss in the biorecognition ability, polymer encapsulation was employed as a strategy to protect immobilized biorecognition elements against SDS treatment. The monomer concentration was optimized to attain a balance between biopreservation and antibody availability for the target antigen capture (FIG. 5F). The polymer-encapsulated AuNRT-NGAL antibody bioconjugates were exposed to different concentrations of NGAL. Although the LSPR shift exhibited by polymer-encapsulated biosensors was ˜50% compared to those without polymer encapsulation, the polymer-encapsulated biosensors exhibited excellent preservation of biorecognition capability after SDS treatment (FIG. 5G). For instance, polymer-encapsulated AuNRT-NGAL antibody bioconjugates exhibited nearly a similar LSPR red shift (˜10 nm) upon exposure to NGAL (5 μg/mL), both before and after SDS treatment, suggesting excellent stability and refreshability. Similar results were observed for different concentrations of NGAL suggesting refreshability of the biosensors over a large range of concentrations (FIG. 5H). The polymer-encapsulated bioconjugates retained nearly 80% of the biorecognition ability after 16 capture/release cycles, which is in stark contrast with less than 20% retained recognition capability of unencapsulated bioconjugates after just three capture/release cycles (FIG. 5I). Finally, as for IgG, the thermal stability of these biosensors was probed by storing the biosensors at different temperatures (room temperature, 40 and 60° C.) for different durations (1, 2, 3, and 7 days). As expected, polymer-encapsulated biosensors retained >60% of biorecognition capability even after storage for 7 days at 60° C., whereas pristine biochips lost more than 80% of the biorecognition capability within 1 day (FIG. 5J). These results attest to the generality of the polymer encapsulation method in preserving and refreshing the biorecognition capabilities of immobilized antibodies.

A facile and universal method is disclosed herein based on in situ polymerization of an organosilica layer for preserving the biorecognition capabilities of immobilized antibodies. Polymer-encapsulated antibodies on plasmonic nanostructures exhibited remarkable stability over multiple capture/release cycles, and thus enabling refreshability of the biochips. The thickness of the polymer layer, controlled by the concentration of the monomers and the polymerization time, plays a critical role in determining the balance between the preservation of the recognition ability of the antibody and the availability of the antibody binding sites for antigen capture. In some embodiments, a plasmonic biosensor is employed as a transduction platform and SDS treatment as a method to overcome the antibody-antigen interaction. In some embodiments, the encapsulation approach comprises other transduction platforms and/or other sensor refreshing methods. More specifically, while the SDS-based sensor refreshing strategy describes an exemplary application embodiment, e.g., primarily to implantables in the oral cavity, the polymer-based preservation method demonstrated herein is universal. The encapsulation-based preservation method demonstrated overcomes a critical challenge in wearable and implantable biosensors and advances the design and implementation of wearable biosensors for long-term monitoring of protein biomarkers.

Materials and Methods

Materials. Ascorbic acid (AA, ≥99.0%), gold(III) chloride trihydrate (HAuCl4.3H2O, ≥99.9%), sodium borohydride (NaBH4, 98%), silver nitrate (>99%), cetyltrimethylammonium bromide (CTAB, ≥99%), cetyltrimethylammonium chloride (CTAC, ≥98%), 3-(mercaptopropyl)trimethoxysilane (MPTMS), trimethoxy(propyl)silane (TMP 5), (3-aminopropyl)trimethoxysilane (APTMS), sodium dodecyl sulfate (>99%) (SDS), albumin from human serum (Mw=65 kDa), sodium hydroxide, phosphoric acid, glycine, 1 M hydrochloric acid and protease from streptomyces griseus were obtained from Sigma-Aldrich. Rabbit IgG, Goat anti-Rabbit IgG (Mw=150 kDa), 1-Ethyl-3-(3-dimethylaminopropyl) carbodiimide (EDC) and N-hydroxysuccinimide (NETS) were purchased from Thermo scientific. Thiol PEF COOH (Mw=5000 g/mol), methoxy PEG thiol (Mw=5000 g/mol) and methoxy PEG silane (Mw=5000 g/mol) was purchased from Jenkem Technology. Neutrophil gelatinase-associated lipocalin (NGAL) and anti-NGAL were purchased from R&D Systems. The phosphate buffer saline (PBS) (10X) buffer was obtained from Thermofisher. Surine (Synthetic urine) was obtained from Dyna Tech Industries. All chemicals were used as received without further modifications.

Synthesis of Gold Nanorods (AuNRs). Gold nanorods were synthesized using a seed-mediated approach. Briefly, first the seed solution was prepared by mixing 9.75 mL of aqueous CTAB solution (0.1 M) and 0.25 mL of HAuCl4 (1 mM) in a 20 mL scintillation vial, followed by the rapid addition of 0.6 mL of ice-cold NaBH4 (10 mM) under vigorous stirring (800 rpm) to yield a brown colored seed solution. Next, the growth solution was prepared by mixing 38 mL of CTAB (0.1 M), 2 mL of HAuCl4 (10 mM), 0.5 mL of silver nitrate (10 mM), and 0.22 mL of ascorbic acid (0.1 M) in the given sequence. The solution was homogenized by gentle stirring which resulted in colorless solution. To the thus formed colorless solution, 0.1 mL of freshly prepared seed solution was added and set aside in dark and static environment for 14 h. Prior to use, the AuNR solution was centrifuged at 10000 rpm for 30 min to remove excess CTAB and re-dispersed in nanopure water (18.2 MΩ.cm).

Synthesis of Gold Nanorattles (AuNRT). First Au nanospheres were synthesized by mixing, in following order, 7 ml of aqueous CTAB solution (0.1 M), 5.25 mL of ascorbic acid (0.1 M), 0.175 ml of freshly synthesized seed solution and 7 ml of HAuCl4 (1 mM) under constant stirring for 1 h. The resulting solution was then centrifuged at 13400 rpm for 30 min. The size of thus formed Au nanospheres was 10 nm. Next 5 ml of the 10 nm nanosphere particle solution was mixed with 45 ml of CTAC solution (20 mM) under stirring at 60° C. for 20 min. Subsequently, 5 mL of AgNO3 (2 mM), 12.5 mL of CTAC (20 mM), and 2.5 mL of ascorbic acid (100 mM) were added under stirring at 60° C. for 4 h. After 4 h, the as-synthesized Au@Ag nanocubes were centrifuged (10000 rpm for 15 min) and redispersed into a 15 mL aqueous solution of CTAC (50 mM). Then to synthesize AuNRT, HAuC14 aqueous solution (0.5 mM) was injected dropwise into the Au@Ag nanocube solution at a rate of 0.5 mL/min (controlled using automated syringe pump) under stirring at 60° C. until the solution turned to blue color or, more precisely, until the LSPR wavelength shifted to ˜665 nm. The AuNRT solution was then centrifuged at 10,000 rpm for 15 min and dispersed in nanopure water for further use.

AuNR-IgG and AuNRT-NGAL antibody bioconjugates preparation. First, EDC and NHS were added to a solution of SH-PEG-COOH in water (37.5 μl, 20 μM), with the same molar ratio as EDC and NHS, followed by shaking for 1 h. Next, the pH of the above solution was adjusted to 7.4 by adding concentrated phosphate buffered saline (10X PBS). Subsequently, rabbit IgG (10 μl, 75 μM) was added to the solution and the resulting solution was then incubated on shaker for 2 h. Then the mixture was filtered to remove any byproduct during the reaction using centrifuge tube with a 50 kDa filter. The final SH-PEG-IgG conjugate solution (0.75 μM) was obtained after washing with PBS buffer (pH 7.4) twice through the filter. AuNR-IgG bioconjugates were prepared by adding 8 μl of the SH-PEG-IgG (concentration ˜1.3 mM in water), 2 μl at a time to a 1 ml solution of twice centrifuged gold nanorods (AuNRs). The amount of SH-PEG-IgG was optimized to obtain maximum coverage of IgG on the AuNR surface by monitoring the red sift in the LSPR. The solution was left for 1 hour on a shaker to complete the conjugation. A similar procedure was employed to prepare AuNRT-NGAL antibody bioconjugates where SH-PEG-NGAL antibody bioconjugates were prepared using NGAL antibody instead of IgG and AuNRT were used instead of AuNRs.

Adsorption of AuNR-IgG and AuNRT-NGAL antibody on glass surface. First, 1×2 cm rectangular slides of glass were cleaned with piranha solution (1:3 (v/v) mixture of 30% H2O2 and H2SO4) followed by extensive rinsing with nanopure water. Please note: Piranha solution is extremely dangerous and thus proper care must be taken while handling and disposing the solution. The cleaned glass slides were then modified with MPTMS, to render thiol functionality, by immersing the glass substrate into 1% (w/v) MPTMS solution in ethanol for 1 h followed by immersion in ethanol for 30 min and thoroughly rinsing with nanopure water and ethanol. AuNR-IgG conjugates were immobilized onto MPTMS-functionalized glass substrates by exposing the glass substrates to AuNR-IgG conjugates solution for 3 h. The modified substrates were rinsed with water and dried under the stream of nitrogen to remove the loosely bound AuNR-IgG bioconjugates. To make sure that the amount of IgG conjugated on the AuNR is consistent for each batch, the same amount and concentration was used of IgG solution (8 μL, 1.3 mM) and AuNR solution (1 mL, optical density of 2.0). Also, the LSPR shift was used to monitor bioconjugation of each batch to ensure similar LSPR red shift (8 nm). Moreover, by controlling the absorption time (3 hours) and optical density of the substrates after incubation (0.8), the same amount of AuNRs was deposited on the glass substrates. Similar procedure was employed to immobilize AuNRT-NGAL antibody bioconjugates on the glass substrates.

Polymer encapsulation and PEGylation. Glass substrates with AuNR-IgG bioconjugates were immersed in 4 mL of 1x PBS (pH 7.4) containing different concentration of TMPS and APTMS for 10 min, followed by rinsing with water and drying under a stream of nitrogen. Subsequently, to avoid nonspecific binding, the substrates were PEGylated by immersing the glass slide in 2 mg/ml methoxy-PEG-thiol (AuNR-IgG glass substrates) and methoxy-PEG-silane (polymer encapsulated AuNR-IgG glass substrates) solution for 2 hours and then rinsed with water and dried under nitrogen stream.

SDS treatment. After polymerization and PEGylation the substrates were exposed to different concentration of Anti-IgG or NGAL in 1x PBS. To release analyte (anti-IgG or NGAL) the substrates were washed by immersion in 3 mL of 0.4 wt.% aqueous solution of SDS in 1x PBS for 5 min. Prior to recapture of analyte on bioconjugates by exposure to different concentration of Anti-IgG, the substrates were rinsed with water and dried in nitrogen.

Characterization. Transmission electron microscopy (TEM) micrographs were recorded on a JEM-2100F (JEOL) field emission instrument operating at an accelerating voltage of 200 kV. Samples were prepared by drying a drop of the solution on a carbon-coated grid, which had been previously made hydrophilic by glow discharge. Atomic force microscopy (AFM) images were obtained using Dimension 3000 (Digital instruments) AFM in light tapping mode.

Extinction Spectra and Raman Spectra Measurements. A Shimadzu UV-1800 spectrophotometer was employed for collecting UV-vis extinction spectra from solution and glass substrates. Raman spectra were obtained using a Renishaw inVia confocal Raman spectrometer mounted on a Leica microscope with 20x objective (NA=0.4) and 785 nm wavelength diode laser (0.5 mW). The spectra were obtained in the range of 600-1800 cm-1 with three accumulations and 10 s exposure time.

Enhancing the Stability of COVID-19 Serological Assay through Metal-Organic Framework Encapsulation

Enzyme-linked immunosorbent assay (ELISA) is widely utilized in serologic assays, including COVID-19, for the detection and quantification of antibodies against SARS-CoV-2. However, due to the limited stability of the diagnostic reagents (e.g., antigens serving as biorecognition elements) and biospecimens, temperature-controlled storage and handling conditions are critical. This issue of reagent stability makes biodiagnostics in resource-limited settings, where refrigeration and electricity are inaccessible or unreliable, particularly challenging. Metal-organic framework (MOF) encapsulation is demonstrated herein as a simple and effective method to preserve the conformational epitopes of antigens immobilized on microtiter plate under non-refrigerated storage conditions. in situ growth of zeolitic imidazolate framework-90 (ZIF-90) was demonstrated to render excellent stability to surface-bound SARS-CoV-2 antigens, thereby maintaining the assay performance under elevated temperature (40° C.) for up to 4 weeks. As an exemplary method embodiment, the preservation of plasma samples from COVID-19 patients using ZIF-90 encapsulation was also demonstrated. The energy-efficient approach demonstrated here will not only alleviate the financial burden associated with cold-chain transportation, but also improve the disease surveillance in resource-limited settings with more reliable clinical data.

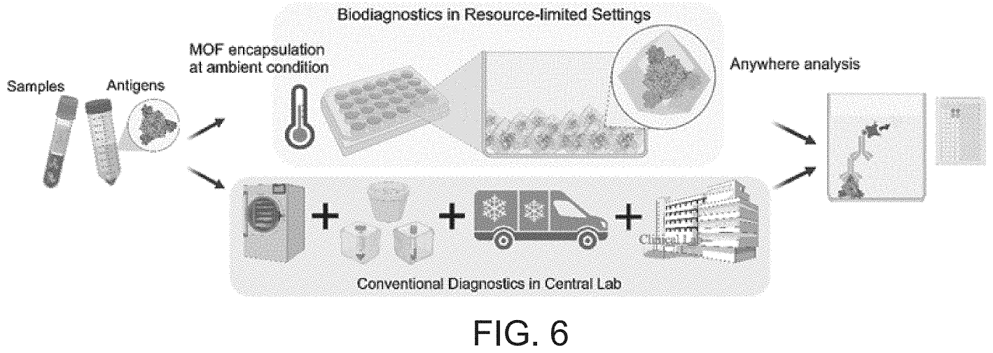

Further demonstrated herein is a zeolitic imidazole framework-90 (ZIF-90) as a simple and effective encapsulation method for preserving the biorecognition capabilities of both SARS-CoV-2 antibodies in patient serum and substrate-immobilized SARS-CoV-2 antigens under elevated temperature and proteolytic conditions. ZIF-90 was in situ grown on SARS-CoV-2 nucleocapsid protein (N protein) and S1 subunit (S1) immobilized on microtiter plate. The SARS-CoV-2 antibodies in patient serum were encapsulated within ZIF-90 crystals by mixing the serum samples with MOF precursors. The biofunctionality of embedded biomolecules were restored through a mild aqueous rinsing step to completely remove ZIF-90 protective layer before implementing the serologic assay. Encapsulation with ZIF-90 significantly improved the stability of surface-bound antigens and antibodies with over 90% of recognition ability after storage at high temperature (up to 60° C.) and exposure to proteases. Overall, the MOF encapsulation method broadly extends the COVID-19 diagnostic, screening and surveillance ability to underserved populations and resource-limited settings (FIG. 6).

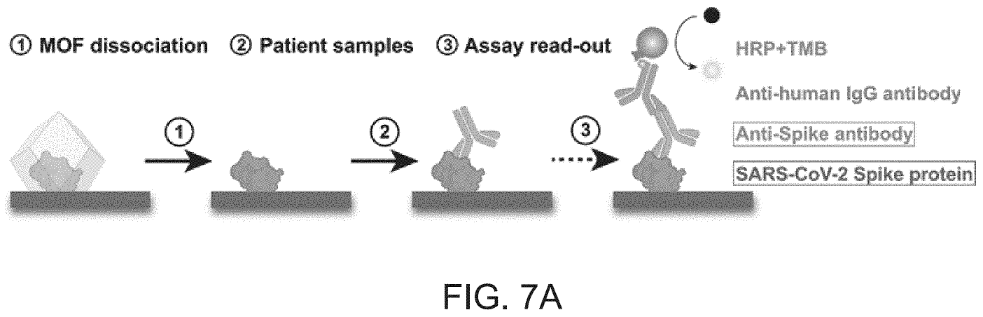

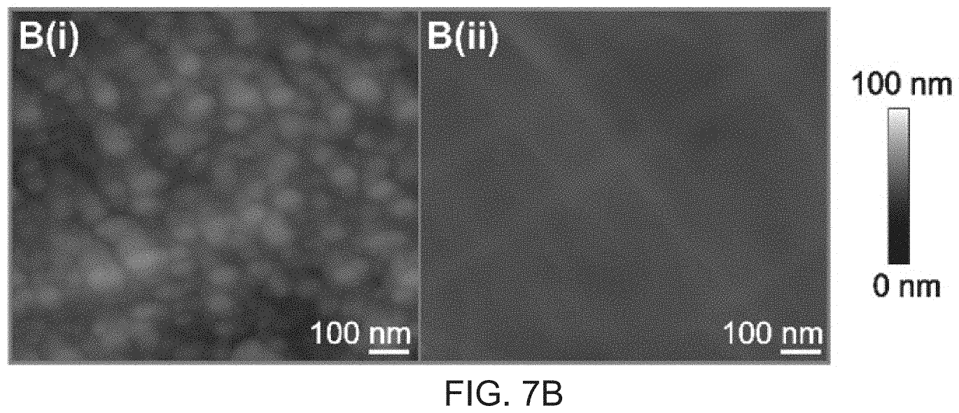

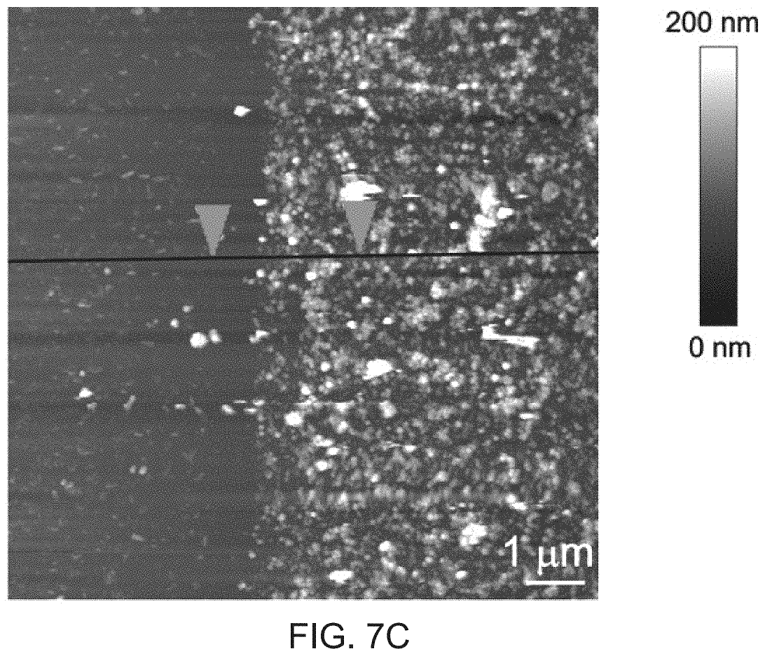

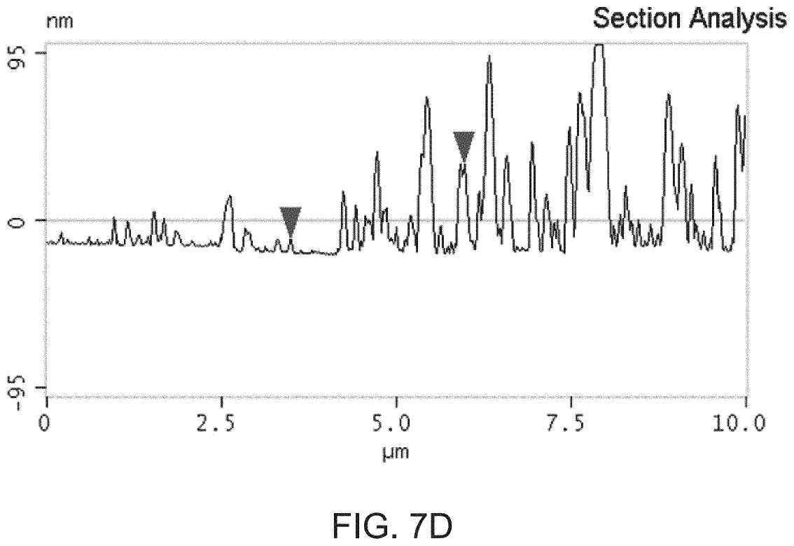

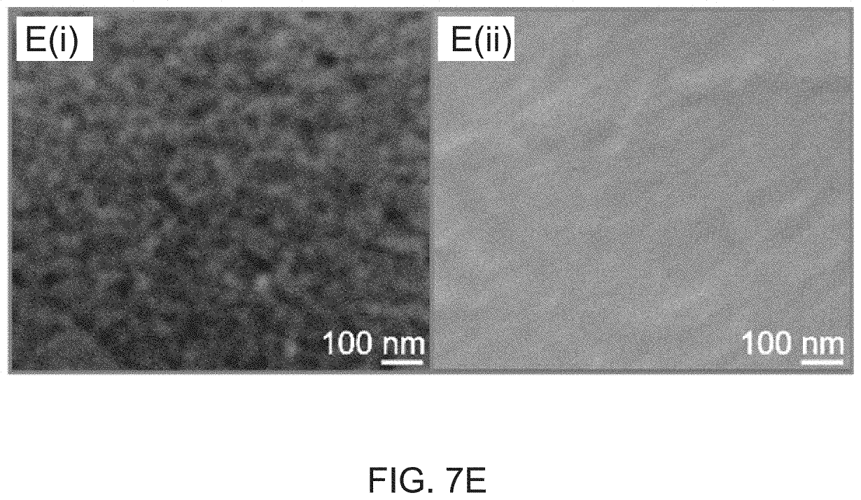

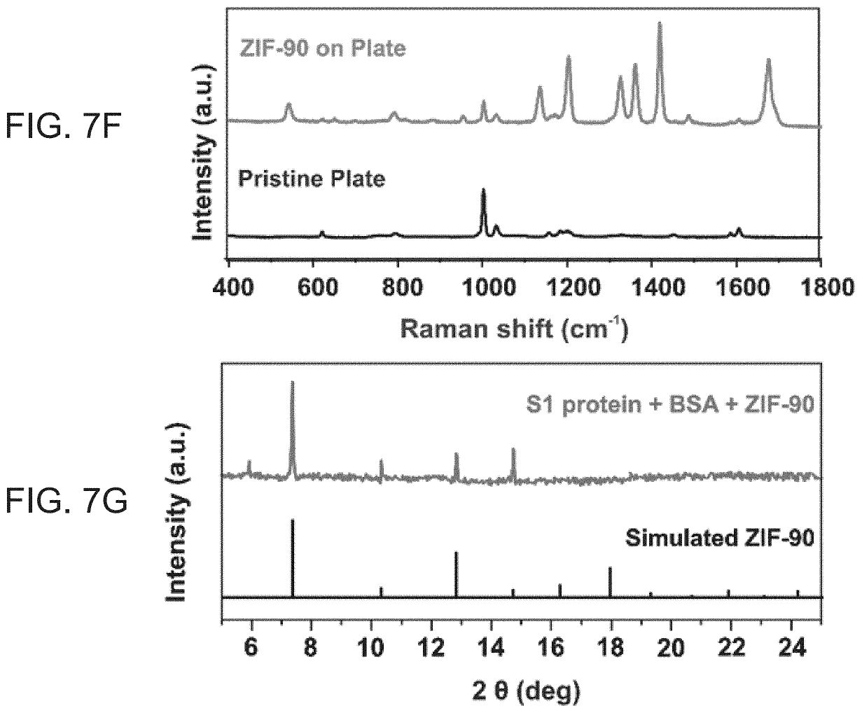

A typical SARS-CoV-2 serologic ELISA involves the immobilization of spike glycoprotein protein (S1 subunit) and nucleocapsid protein (N protein) as antigens, selective capture of corresponding antibodies in patient serum, followed by binding of secondary antibodies and labeled by enzymatic reporters (FIG. 7A). N protein is the most abundant protein in SARS-CoV-2 virion. S protein, comprised of two subunits (S1 and S2), is a type-I transmembrane glycoprotein that plays an important role in mediating viral infection, where the S1 subunit binds to the cellular receptors through its receptor-binding domain (RBD). The antibodies against N protein are usually more abundant compared to those against S1, while the latter better correlate with the protection against the disease compared to the former.

To preserve the surface-bound N and S1 antigens for bioassays, the feasibility of in situ growth and dissociation of ZIF-90 was first investigated on the surface-bound antigens. Owing to the rich functional groups, proteins serve as nucleating sites for the fast nucleation and growth of ZIF-90 crystals. ZIF-90 crystals render tight encapsulation of the antigens, and minimize the changes in their secondary and tertiary structure even under harsh environmental conditions. The ZIF-90 protective layer was formed by incubating the antigen-coated microtiter plate with the precursor solution (a mixture of zinc nitrate and 2-imidazolatecarboxyaldehyde) for one hour. After storing the ZIF-90 protected plate for a desired duration at desired temperature, the protective layer was removed by EDTA/phosphate buffer solution (pH˜5.4) before performing the bioassay (see Experimental section for details). The ZIF-90 film was characterized by atomic force microscope (AFM) and scanning electron microscope (SEM). AFM images revealed distinct morphology of antigen-coated plate before and after ZIF-90 coating (FIG. 7B). With ZIF-90 coating, a dense grainy morphology (FIG. 7B, (i)) was observed and the AFM scratch test on silicon indicated the thickness of ZIF-90 layer to be 40±5 nm (FIGS. 7C and 7D). Scanning electron microscope (SEM) images further confirmed the distinct morphology of MOF-coated plate (FIG. 7E, (i)) compared to the plate without MOF coating (FIG. 7E, (ii)). The growth and removal of ZIF-90 layers was also confirmed by Raman spectroscopy (FIG. 7F). Raman spectra obtained from ZIF-90 coated plate exhibited bands originating from the 2-imidazolatecarboxyaldehyde ring vibration at 1135 and 1202 cm-1. Other Raman bands were observed at 1325 (δH—CO), 1361 (SC—H) and 1419 cm-1 (νC2—N1). Raman band at 1675 cm-1 obtained from plate with ZIF-90 coated antigens are ascribed to amide moieties of the proteins. The shift in the amide band from 1650-1655 cm-1 to 1675 cm-1 suggests a slight change of coordination environment due to the interactions between protein and ZIF-90. X-ray diffraction (XRD) also confirmed the formation of ZIF-90 crystals on the antigen-coated plate (FIG. 7G). XRD peak positions of the antigen-encapsulating ZIF-90 crystals on silicon substrate were mostly identical with the simulated XRD pattern of ZIF-90, which further confirmed the in-situ formation of ZIF-90 on the ELISA plate.

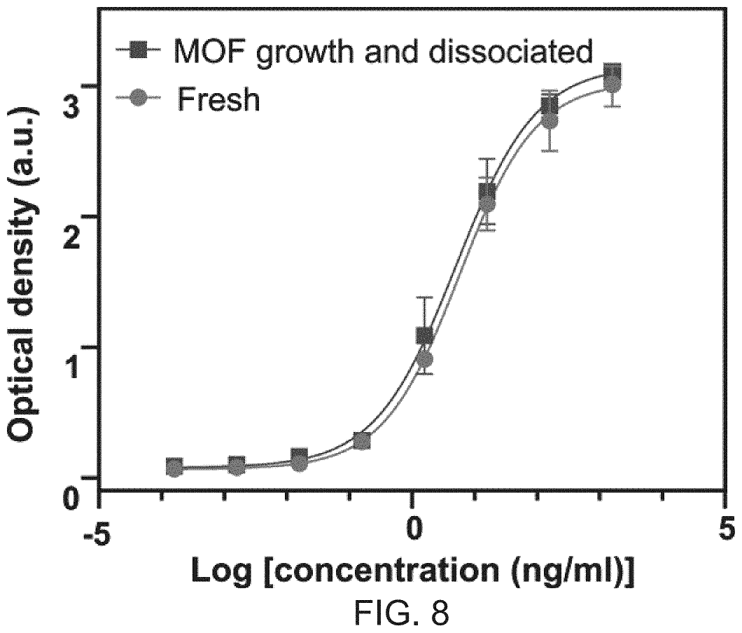

Initially, the effect of the growth and removal process of ZIF-90 was investigated on specificity and sensitivity of SARS-CoV-2 serological assay. To assess the preservation efficacy of ZIF-90 coatings, the limit-of-detection (LOD) and signal intensity (i.e. optical density at 450 nm) attained from microtiter plates stored in different conditions were compared with those obtained from plates stored under “gold standard” refrigerated condition (i.e. stored at −20° C. with sucrose protection). LOD of ELISA, a commonly used metric for measuring assay sensitivity, was defined as the concentration corresponding to the mean+3×standard deviation (σ) of the lowest concentration point (or blank). To explore the possible influence of MOF coating and removal process, ZIF-90 layer was grown on antigen-coated plate, followed by removal of the overlayer. Serial dilution of antibodies with known concentration were employed as standards. The calculated LOD deduced from the standard curve obtained using freshly prepared (control) plate and ZIF-90 treated plate was 7.06 pg/ml and 7.54 pg/ml (FIG. 8), respectively, indicating that the encapsulation and removal processes have negligible effect on the biofunctionality of surface-bound antigens.

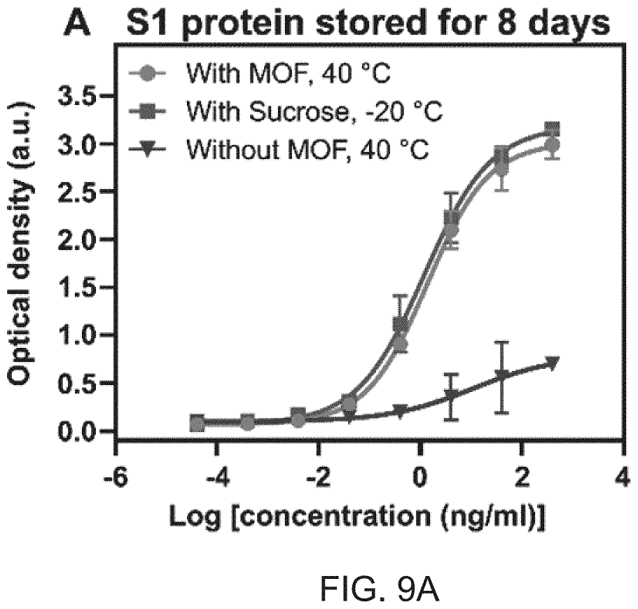

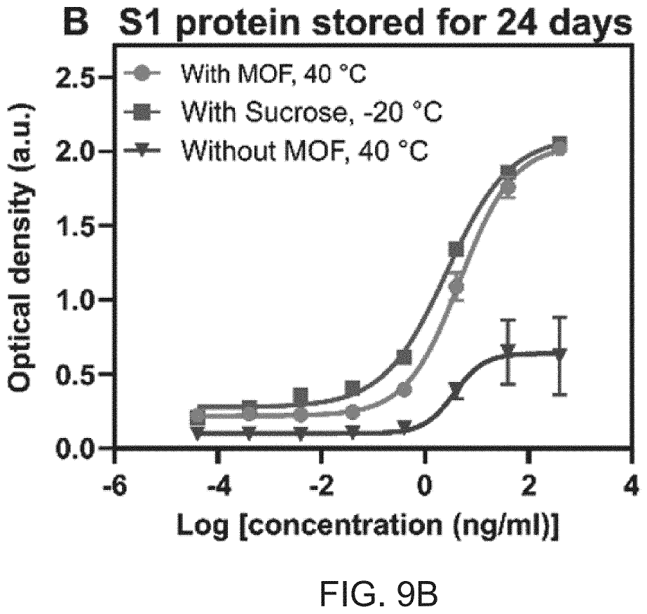

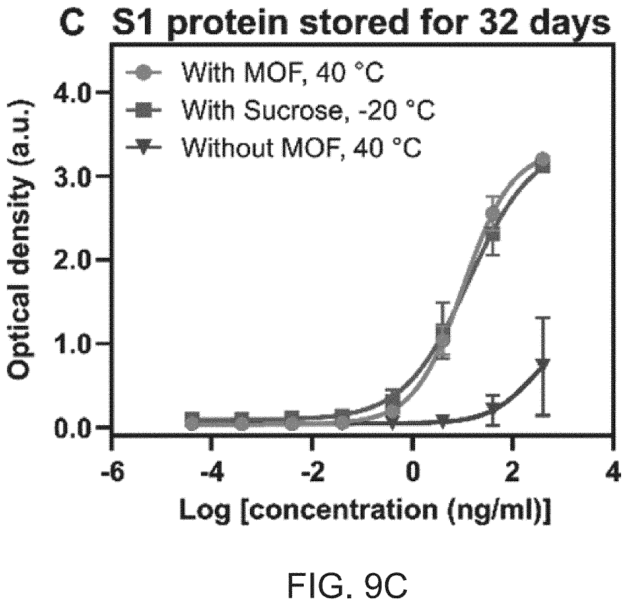

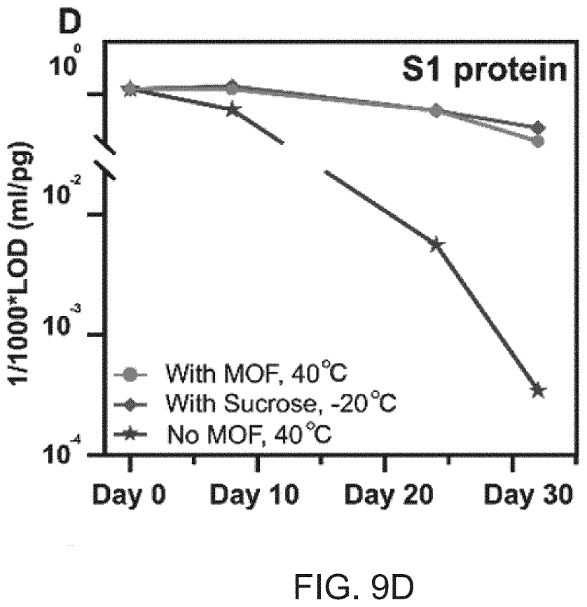

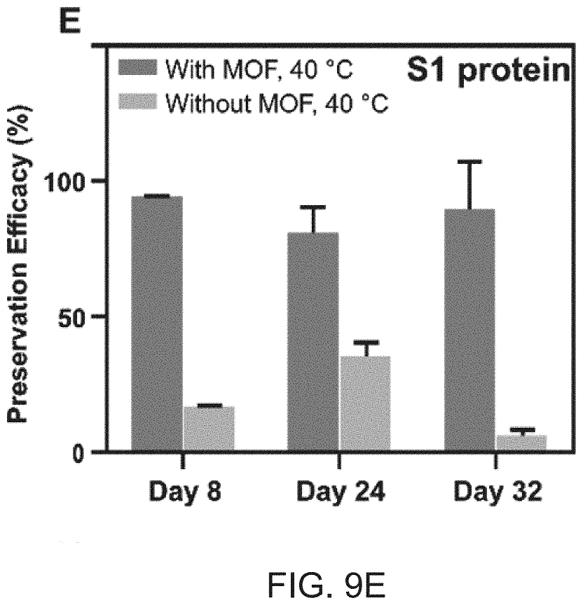

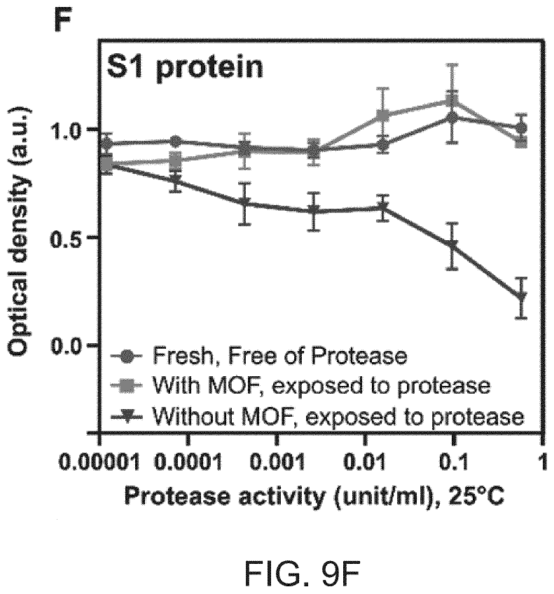

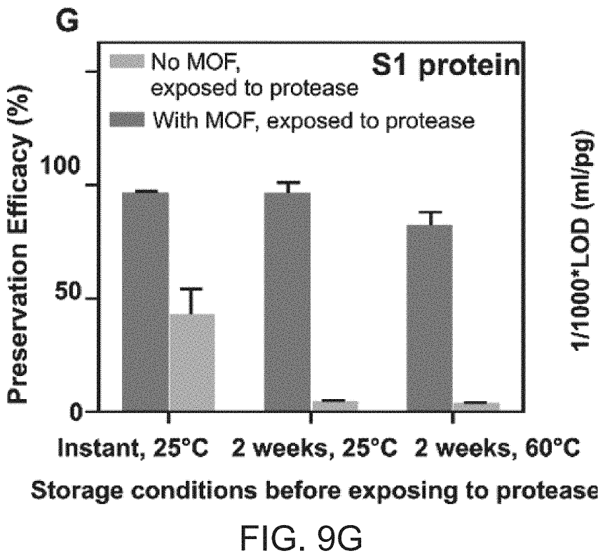

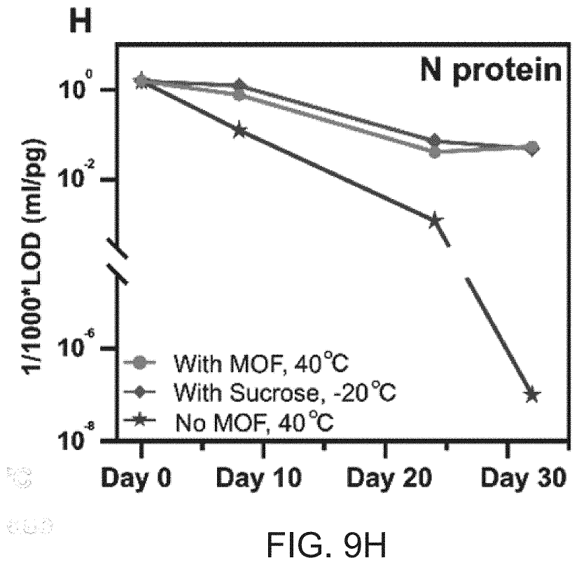

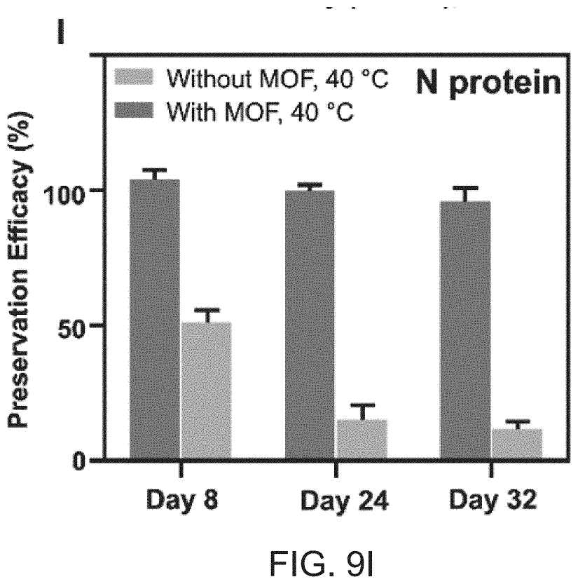

Next, the efficacy of ZIF-90 in preserving the surface-bound SARS-CoV-2 antigens against harsh environmental conditions was investigated (at 40° C. up to 32 days, or exposure to proteases), simulating transport and long-term storage conditions that would normally lead to loss of conformational epitopes of these biorecognition elements. The S1 protein coated plates with and without ZIF-90 encapsulation were stored at 40° C. for 8 days, 24 days and 32 days, and plates stored with sucrose protection at −20° C. were used as “gold standard” reference (FIGS. 9A, 9B and 9C). After 32 days of storage at 40° C., the LOD of ZIF-90 protected microtiter plate was found to be 39.4 pg/ml for S1 protein, which is comparable to the LOD of the plates stored under refrigeration (14.2 pg/ml). The LOD of microtiter plate with ZIF-90 encapsulation was found to be around 80-fold lower for S1 protein compared to that without ZIF-90 protection after storage at 40° C. for 32 days (FIG. 9D). the preservation efficacy of ZIF-90 was calculated by comparing the OD values obtained from linear region of the standard curve under different storage conditions (FIGS. 9A, 9B and 9C). Preservation efficacy (%) is calculated as the percentage of the OD obtained from a restored microtiter plate after storage under different conditions compared to the OD obtained with the same batch of freshly fabricated microtiter plate. After 8 days of storage, ZIF-90 encapsulated plates exhibited only about 5% loss in sensitivity for surface-bound S1 proteins, which is significantly lower compared to the nearly 50% loss in sensitivity for plates without ZIF-90 (FIG. 9E). Significantly, after storage for 32 days, ZIF-90 protected microtiter plates exhibited sensitivity close to that of the plates stored at −20° C. with sucrose protection, with a preservation efficacy of 93%. In contrast, plates stored under identical conditions without MOF protection exhibited nearly 90% loss in sensitivity (FIG. 9E).

In addition to storage at elevated temperature, the efficacy of ZIF-90 in preserving the surface-bound antigens against proteolytic conditions was assessed (FIG. 9F and 9G). Proteases cause antigen degradation by cleaving the peptide bonds, thus compromising their biorecognition ability. After exposure to protease solution (protease from Streptomyces griseus) at various concentrations for 60 mins, it was noted that surface-bound S1 protein retained less than 20% of biorecognition capability (as determined from the OD values in the linear region of the standard curve) for protease activity of 0.525 unit/ml (FIG. 9F). Remarkably, surface-bound S1 protein with ZIF-90 protection retained over 90% biorecognition ability under protease activity (0.525 unit/ml), which is similar to pristine surface-bound S1 protein without protease treatment (FIG. 9F). Furthermore, the protease shielding ability of ZIF-90 after thermal treatment was investigated. Surface-bound S1 protein stored at room temperature and 60° C. for 2 weeks were exposed to proteases for 1 hour. Protease treatment (0.0875 unit/ml) of the unprotected surface-bound S1 protein exhibited less than 50% of biorecognition capacity (FIG. 9G). ZIF-90 encapsulated antigens, after storage at room temperature for 2 weeks followed by protease treatment for 1 hour, retained a recognition capability of 96%, whereas antigens subjected to identical harsh conditions without ZIF-90 encapsulation exhibited nearly complete loss (less than 5% retained) of biorecognition ability (FIG. 9G). Surface-bound S1 protein stored at 60° C. for 2 weeks and exposed proteases for 1 hour retained above 82% of recognition capability with ZIF-90 protection, while less than 5% of recognition ability was retained without ZIF-90 protection (FIG. 9G). These stress tests indicate that ZIF-90 is capable of shielding S1 protein from both proteases and elevated temperature.

In addition to S1 protein, the stabilization of N protein was investigated using similar strategy. For surface-bound N protein stored at 40° C. for 32 days, the LOD of ZIF-90 protected microtiter plates was calculated to be 18.8 pg/ml, which is comparable to “gold standard” refrigeration method (20.8 pg/ml) (FIG. 9H). Furthermore, ZIF-90 protected surface-bound N protein retained over 90% of recognition ability after storing at 40° C. for 32 days, whereas less than 12% of recognition capability was retained without ZIF-90 protection (FIG. 9I). The consistent results of S1 protein and N protein indicate the universality of the ZIF-90 encapsulation in preserving the surface-bound antigens on microtiter plates.

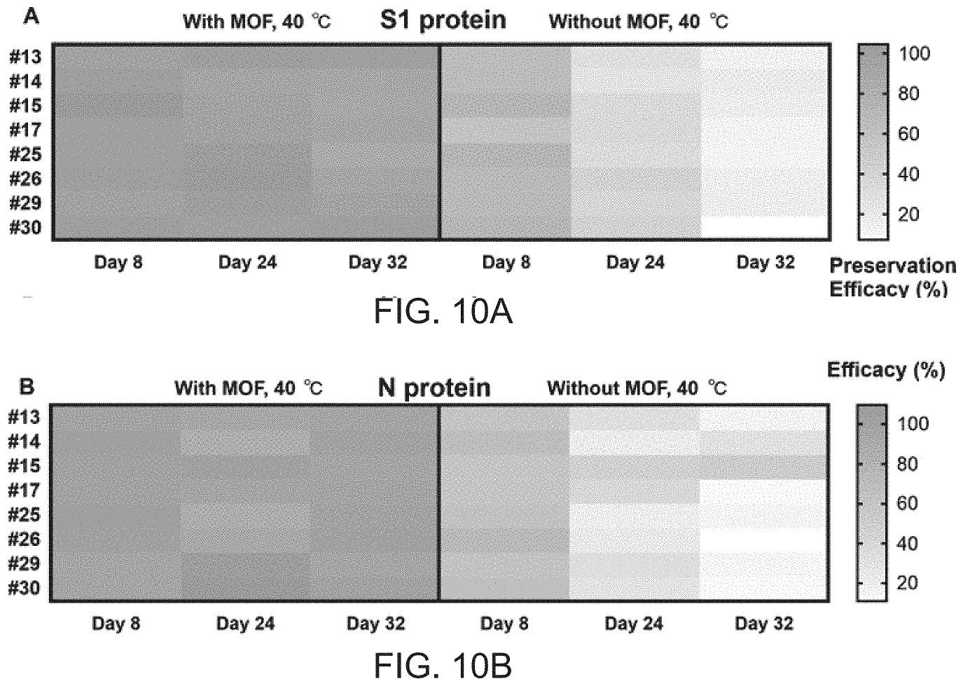

Next, the applicability of ZIF-90 encapsulated microtiter plates in analyzing patient plasma samples was demonstrated. Compared to purified antibodies (employed as standard in the experiments described above), human plasma sample represents a complex biological matrix, comprised of various biomolecules such as antibodies, enzymes and metabolites that interfere with diagnostics assays. The ability of ZIF-90 protected surface-bound antigens to recognize antibodies was evaluated in plasma samples obtained from COVID-19 patients. Eight plasma samples from COVID-19 patients (#13, #14, #15, #17, #25, #26, #29 and #30) were tested for antibodies against SARS-CoV-2 using S1 protein and N protein (antigens) as biorecognition elements. Surface-bound S1 protein on ELISA plates with and without ZIF-90 protection were stored at 40° C. for 8 days, 24 days and 32 days. Plates protected with sucrose at −20° C. were employed as reference. Before testing, all patient samples were diluted with 6000-fold in phosphate buffered saline (PBS), to ensure that the final testing concentration is in the linear range of standard curve (standard curves shown in FIGS. 9A, 9B and 9C). ZIF-90 protected antigens (S1 protein and N proteins) immobilized on microtiter plate retained above 95% of preservation efficacy after 8 days and above 90% of preservation efficacy after 32 days stored at 40° C. (FIGS. 10A and 10B). In stark contrast, antigens on the plates without ZIF-90 protection show less than 60% of preservation efficacy after 8 days of storage and less than 15% of preservation efficacy after storage for 32 days at 40° C. (FIGS. 10A and 10B). Such stable biodiagnostic performance of ZIF-90 protected microtiter plates coated with desired antigens indicate the feasibility of harnessing this encapsulation approach in deploying the serological assay in resource-limited settings.

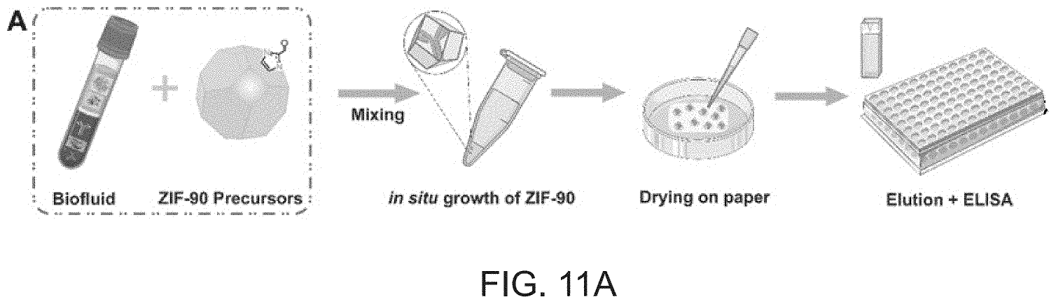

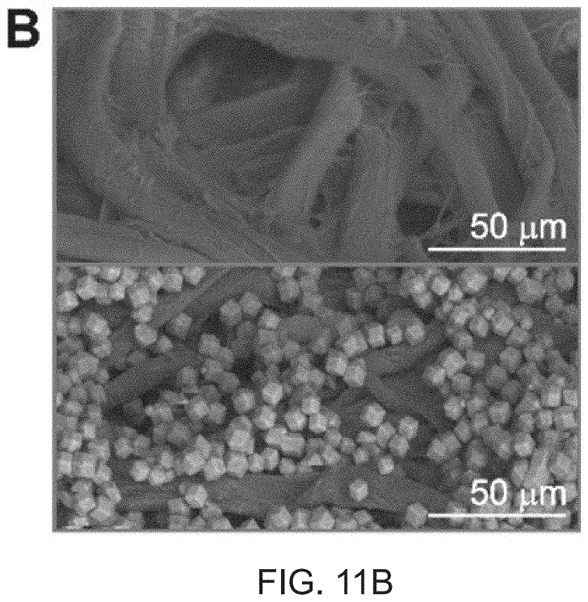

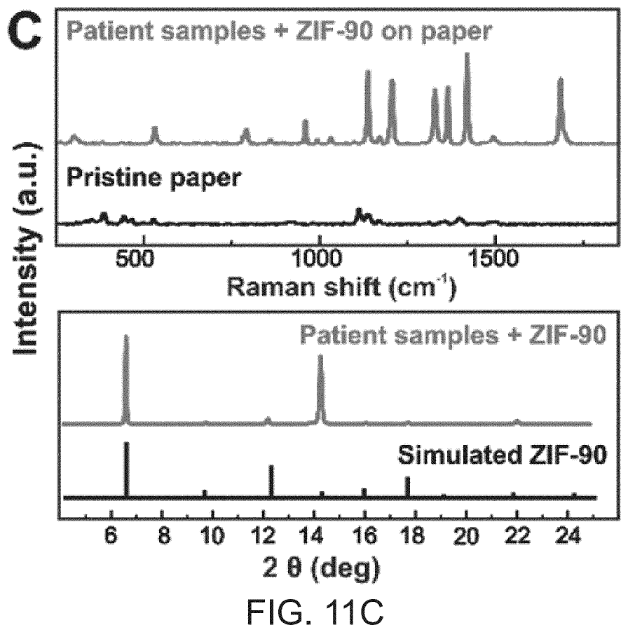

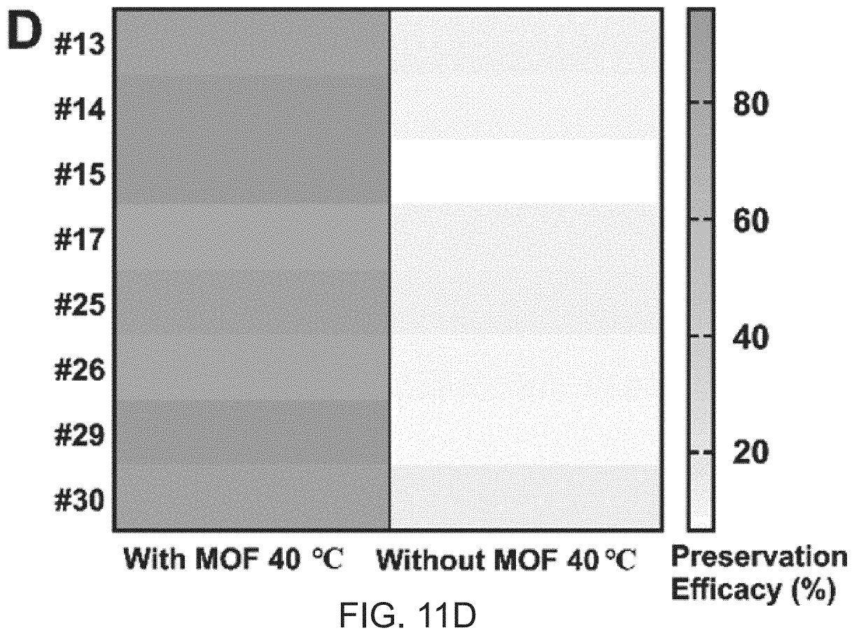

Finally, it was determined that ZIF-90 enabled plasma stabilization as a complementary approach to deploy biodiagnostics in resource-limited settings. The preservation and testing methods are based on known protocols with slight modifications. As illustrated in FIG. 11A, plasma samples from COVID-19 patients were first mixed with 2-imidazolatecarboxyaldehyde (200 mM) followed by zinc nitrate solution (200 mM). After 40 mins of incubation at room temperature, plasma containing SARS-CoV-2 antibodies encapsulated in ZIF-90 crystals were collected by drying the solution on Whatman 903 paper strip (FIG. 11A). The ZIF-90 nanocrystals encapsulating the plasma components were characterized by SEM, Raman and XRD. After encapsulation of patient plasma samples, the SEM images exhibit sodalite morphology (with particle size of 2 μm-10 μm) (FIG. 11B). Raman bands at 1137 cm-1, 1205 cm-1 and 1329-1419 cm-1 correspond to C═O stretching vibration of 2-imidazolatecarboxyaldehyde and the peak at 1675 cm-1 are ascribed to amide bonds of the encapsulated proteins (FIG. 11C). XRD pattern of plasma-embedded ZIF-90 crystals display the characteristic peaks corresponding to pristine ZIF-90 crystals (FIG. 11C).

The thermal stability of ZIF-90 encapsulated plasma sample was evaluated by storing plasma samples with and without ZIF-90 encapsulation at 40° C. for over 3 weeks, which serves as a surrogate for harsh transport/storage condition. The preservation efficacy was then calculated by comparing the amounts of antibodies detected with ZIF-90 protection with that in the pristine samples stored under refrigeration. For all 8 patients ((#13, #14, #15, #17, #25, #26, #29 and #30), the samples with ZIF-90 encapsulation resulted in more than 80% preservation efficacy after 3 weeks stored at 40° C., whereas less than 15% preservation efficacy was observed in samples without ZIF-90 encapsulation (FIG. 11D). Overall, ZIF-90 encapsulation method presented in this work serves as a simple and robust strategy for preserving biodiagnostic capability of surface-bound antigens and antibodies in patient samples by confining the conformational structure of biomolecules on substrates and in biofluids.

The ZIF-90 encapsulation of antigens immobilized on a microtiter plate and its role as an exoskeleton in protecting the biorecognition ability of antigens against harsh environment conditions is herein demonstrated (including elevated temperature (up to 60° C.), protease and long-term storage without refrigeration). The ZIF-90 protected surface-bound antigens at elevated temperature retained above 80% biorecognition capability after storage at 40° C. for over one month, which is comparable to existing “gold standard” methods (storage at −20° C. with sucrose protection). In addition, it was demonstrated that ZIF-90 encapsulation preserves SARS-CoV-2 antibodies in patient plasma samples at 40° C. for over 3 weeks, with preservation efficacy comparable to that of the refrigeration method. The high thermal stability of antigens and antibodies rendered by ZIF-90 encapsulation extend the benefits of biodiagnostics to resource-limited settings and underserved populations. As described herein, MOF encapsulation effectively reduces or eliminates the need for cold-chain in biodiagnostics and decreases the reliance on centralized labs, improving the overall effectiveness in utilizing the advances in ultrasensitive biodiagnostics in controlling the outbreaks of infectious diseases and early detection and monitoring of other pathological conditions.

Materials and Methods

Chemicals. Recombinant SARS-CoV-2 Nucleocapsid protein (Cat.# 230-30164) and Recombinant SARS-CoV-2 S1 subunit (Cat.# 230-01102) were purchased from Raybiotech, Inc. SARS-CoV-2 Nucleocapsid (N) protein (Rabbit) antibody (Cat.# 600-401-MS4) and SARS-CoV-2 spike (S1) protein (Rabbit) antibody (Cat.# 600-401-MS8), biotinylated anti-human IgG (H&L) (Rabbit) (Cat.# 609-4617), and biotinylated donkey anti-rabbit IgG (Cat.# 616-4102) were purchased from Rockland, Inc. Streptavidin-HRP, and TMB substrate were purchased from R&D System. Zinc nitrate, 2-imidazolecarboxaldehyde (ICA), ethylenediaminetetraacetic acid (EDTA), Tween 20, sodium phosphate monobasic, sodium phosphate dibasic, sodium formate and protease (protease from Streptomyces griseus) were purchased from Sigma-Aldrich. Patient samples were obtained from the Washington University Institutional Review Board, and written informed consent was obtained from all patients (IRB 202003186). All patients have been tested positive for SARS-CoV-2 with RT-PCR before serum collection.

Encapsulation of proteins on microtiter plate with ZIF-90 films. Microtiter plate was first coated with 2 μg/ml S1 protein or N protein in PBS at room temperature for overnight and blocked with 1% BSA in PBS for 3 hours. For ZIF-90 encapsulation, 2-imidazolecarboxaldehyde (ICA) (200mM) and zinc nitrate solution (200 mM) was simultaneously added into each well and agitated for two hours. Subsequently after aspiration of liquid and drying with a stream of nitrogen, the plates were stored at 25° C. and 40° C. for different time intervals and subjected to other harsh conditions as described in the main text.

Encapsulation of proteins in patient serum with ZIF-90 crystals. Patient serum (1.2 μl) were first diluted 60-times with PBS before being mixed with 2-imidazolecarboxaldehyde (320 μl) and 80 μl of zinc nitrate solution simultaneously. The final concentration of 2-imidazolecarboxaldehyde and zinc nitrate is 200 mM. After 40 mins of incubation at room temperature (20-23° C.), mixture solution was pipetted onto Whatman 903 paper strip, followed by air-drying. The ratio between fluid volume and area of Whatman paper was maintained around 50 μl/cm2 to avoid liquid leakage from paper strip. After air-drying, strips were sealed in Petri dishes and stored at 40° C. for 3 weeks.

Protein recovery and ELISA. To recover embedded proteins from ZIF-90 crystals, MOF dissociation buffer (0.1 M phosphate buffer with 200 mM EDTA and 0.1% Tween20 at pH 5.4) was added to each of the wells and subjected to orbital shaking for 15 minutes, followed by aspirating the buffer and washing with PBST (1X PBS, 0.05% Tween20). Serial dilutions of rabbit anti SARS-CoV-2 Spike or Nucleocapsid protein with known concentration were used as standards and applied on the eluted plates for 2 hours. The concentrations of anti-nucleocapsid (N) protein range from 160 fg/ml to 1.6 mg/ml, while the concentrations of anti-spike protein antibody range from 40 fg/ml to 0.4 mg/ml. After washing with PBST, plate was incubated with biotin labelled anti rabbit IgG (1:2000 in 1% BSA-PBST) for another 2 hours, followed by the addition of HRP-labelled streptavidin for 20 min. 100 μl of substrate solution was subsequently added to each well and the reaction was stopped with 50 μl H2SO4 (2 N). Optical density of each well was determined immediately using a microplate reader set to 450 nm. The assay for patient serum samples was similar except that biotin-labelled anti human IgG was used as secondary antibody.

To recover embedded proteins from ZIF-90 crystals, the paper strips were eluted in a cuvette containing 1 ml of elution buffer (0.1 M phosphate buffer with 200 mM EDTA and 0.1% Tween20 at pH 5.4) with gentle shaking for 45 minutes. The elution solution was then assayed by ELISA for SARS-CoV-2 specific antibody. Microtiter plate was coated with 2 μg/ml S1 protein or N protein in PBS at room temperature for overnight and blocked with 1% BSA in PBS for 3 hours before being applying the eluted patient serum. Subsequent assay steps are similar to the abovementioned protocol.

Material Characterization. Atomic force microscopy (AFM) images were collected by Dimension 3000 AFM (Digital instruments) in light tapping mode. The X-ray diffraction (XRD) measurements of the samples were recorded on a Bruker D8-Advance X-ray powder diffractometer using Cu Kα radiation (λ=1.5406 Å) with scattering angles (2θ) of 5-25°. The Raman spectra were obtained using a Renishaw inVia confocal Raman spectrometer mounted on a Leica microscope with a 50X objective and a 785 nm wavelength diode laser was employed as the illumination source. SEM images were obtained using Thermo Scientific Quattro S Environmental Scanning Electron Microscope (ESEM).

Abbreviations: AFM: atomic force microscopy, APTMS: (3-aminopropy) trimethoxysilane, AuNRs: gold nanorods, AuNRT: gold nanorattle, HSA: human serum albumin, LOD: limit of detection, LSPR: localized surface plasmon resonance, MPTMS: 3-mercaptopropyl-trimethoxysilane, NGAL: neutrophil gelatinase-associated lipocalin, PA: phosphoric acid, PEG: poly(ethylene glycol), SDS: sodium dodecyl sulfate, SERS: surface-enhanced Raman scattering, SH-PEG: thiol-terminated poly(ethylene glycol), TEM: transmission electron micrograph, TMPS: trimethoxy(propyl)silane.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that vary depending upon the desired properties sought to be obtained by a particular embodiment. In some embodiments, the numerical parameters are be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) are construed to cover both the singular and the plural, unless specifically noted otherwise. In some embodiments, the term “or” as used herein, including the claims, is used to mean “and/or” unless explicitly indicated to refer to alternatives only or to refer to the alternatives that are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and may also cover other unlisted steps. Similarly, any composition or device that “comprises,” “has” or “includes” one or more features is not limited to possessing only those one or more features and may cover other unlisted features.

All methods described herein are performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member is referred to and claimed individually or in any combination with other members of the group or other elements found herein. One or more members of a group are included in, or deleted from, a group for reasons of convenience or patentability. When any such inclusion or deletion occurs, the specification is herein deemed to contain the group as modified thus fulfilling the written description of all Markush groups used in the appended claims.

To facilitate the understanding of the embodiments described herein, a number of terms are defined below. The terms defined herein have meanings as commonly understood by a person of ordinary skill in the areas relevant to the present disclosure. Terms such as “a,” “an,” and “the” are not intended to refer to only a singular entity, but rather include the general class of which a specific example may be used for illustration. The terminology herein is used to describe specific embodiments of the disclosure, but their usage does not delimit the disclosure, except as outlined in the claims.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A biosensor comprising:
a plasmonic nanostructure; and
at least one biorecognition element, wherein the biorecognition element is encapsulated with at least one of an organosilica polymer layer or a metal organic framework (MOF).

2. The biosensor of claim 1, wherein the MOF comprises zeolitic imidazolate framework-90 (ZIF-90).

3. The biosensor of claim 1, wherein the at least one organosilica polymer layer comprises (3-aminopropyl) trimethoxysilane (APTMS) monomers, trimethoxy(propyl)silane (TMPS) monomers, or a combination thereof.

4. The biosensor of claim 1, wherein the biorecognition element is anchored to the plasmonic nanostructure via a flexible linker comprising poly(ethylene glycol) (PEG).

5. The biosensor of claim 1, wherein the plasmonic nanostructure is selected from the group consisting of a gold nanorod and a gold nanorattle.

6. The biosensor of claim 1, wherein the biorecognition element comprises an antibody. The biosensor of claim 1, wherein the biorecognition element comprises an antigen.

8. A method for synthesizing a refreshable biosensor, the method comprising:
immobilizing at least one biorecognition element on a plasmonic nanostructure; and
encapsulating the at least one biorecognition element with at least one of an organosilica polymer layer via in situ polymerization or a metal organic framework via in situ crystallization.

9. The method of claim 8, wherein the MOF comprises zeolitic imidazolate framework-90 (ZIF-90).

10. The method of claim 8, wherein the organosilica polymer layer comprises (3-aminopropyl) trimethoxysilane (APTMS) monomers, trimethoxy(propyl)silane (TMPS) monomers, or a combination thereof.

11. The method of claim 8, wherein immobilizing the at least one biorecognition element on the plasmonic nanostructure comprises anchoring the at least one biorecognition element to the plasmonic nanostructure via a flexible linker comprising poly(ethylene glycol) (PEG).

12. The method of claim 8, wherein the plasmonic nanostructure is selected from the group consisting of a gold nanorod and a gold nanorattle.

13. The method of claim 8, wherein the biorecognition element comprises an antibody.

14. The method of claim 8, wherein the biorecognition element comprises an antigen.

15. A method for refreshing a biosensor, the method comprising:
exposing the biosensor to a target analyte, wherein the biosensor comprises at least one biorecognition element immobilized on a plasmonic nanostructure, and wherein the at least one biorecognition element is encapsulated with at least one of an organosilica polymer layer or a metal organic framework (MOF);
rinsing the biosensor with an aqueous sodium dodecyl sulfate solution; and
re-exposing the biosensor to the target analyte.

16. The method of claim 15, wherein the MOF comprises a zeolitic imidazolate framework-90 (ZIF-90).

17. The method of claim 15, wherein the organosilica polymer layer comprises (3-aminopropyl) trimethoxysilane (APTMS) monomers, trimethoxy(propyl)silane (TMPS) monomers, or a combination thereof.

18. The method of claim 15, wherein the biorecognition element is immobilized on the plasmonic nanostructure via a flexible linker comprising poly(ethylene glycol) (PEG).

19. The method of claim 15, wherein the plasmonic nanostructure is selected from the group consisting of a gold nanorod and a gold nanorattle.

20. The method of claim 15, wherein the biorecognition element is selected from the group consisting of an antibody and an antibody.
